(19) United States
(12) Patent Application Publication
(10) Pub. No.: US 2014/0234420 A1
(43) Pub. Date: Aug. 21, 2014

(54) Method of Treating Skin Disorders using Nanoscale Delivery Devices and Transdermal Enhancing Compositions

(71) Applicants: Omathanu P. Perumal, Brookings, SD (US); Ranjith Kumar Averineni, Brookings, SD (US)

(72) Inventors: Omathanu P. Perumal, Brookings, SD (US); Ranjith Kumar Averineni, Brookings, SD (US)

(21) Appl. No.: 13/967,274

(22) Filed: Aug. 14, 2013

Related U.S. Application Data
(63) Continuation-in-part of application No. 13/404,536, filed on Feb. 24, 2012.
Application No. 13/404,536 is a continuation-in-part of application No. 12/991,872, filed on Dec. 13, 2010, now Pat. No. 8,669,225.
Application No. 12/991,872 is a continuation-in-part of application No. 13/404,392, filed on Feb. 24, 2012, now Pat. No. 8,697,098.
(60) Provisional application No. 61/683,185, filed on Aug. 14, 2012.
(60) Provisional application No. 61/683,203, filed on Aug. 14, 2012.
(60) Provisional application No. 61/446,934, filed on Feb. 25, 2011.
(60) Provisional application No. 61/127,134, filed on May 9, 2008.
(60) Provisional application No. 61/446,931, filed on Feb. 25, 2011.

(51) Int. Cl. A61K 47/42; A61K 9/51; A61K 9/107; A61K 31/519

(52) U.S. Cl. CPC A61K 47/42; A61K 31/519; A61K 9/5169; A61K 9/1075
USPC 424/489; 514/559; 514/714; 514/249; 514/163; 424/705; 514/29; 514/24; 514/569; 514/164; 514/161

(57) ABSTRACT

The present invention describes methods and compositions for treating diverse dermatological conditions, including acne and psoriasis, using zein containing nanocarrier devices for topical delivery of methotrexate, retinoic acid and benzoyl peroxide to select targets in the skin.

### CROSS REFERENCE TO RELATED APPLICATIONS

This application is a Continuation-In-Part of U.S. application Ser. No. 13/404,536, filed Feb. 24, 2012, which claims benefit under 35 U.S.C. §119(e) to U.S. Provisional Application No. 61/446,934, filed Feb. 25, 2011. This application is a Continuation-In-Part of U.S. application Ser. No 12/991,872, filed Dec. 15, 2010, which is a National Phase Entry of PCT/US2009/002935, filed May 11, 2009, which claims benefit under 35 U.S.C. §119(e) to U.S. Provisional Application No. 61/127,134, filed May 9, 2008. This application is a Continuation-In-Part of U.S. application Ser. No. 13/404,392, filed Feb. 24, 2012, which claims benefit under 35 U.S.C. §119(e) to U.S. Provisional Application No. 61/446,035, filed Feb. 25, 2011. This application also claims benefit under 35 U.S.C. §119(e) to U.S. Provisional Application Nos. 61/683,185 and 61/683,203, both filed Aug. 14, 2012, which are incorporated by reference herein in their entireties.

### BACKGROUND OF THE INVENTION

1. Field of the Invention

The present invention relates generally to topical delivery, and more specifically to a) protein nanocarriers for topical delivery of medicaments such as methotrexate (MTX), retinoic acid and benzoyl peroxide (BPO) to the skin for treating various dermatological conditions, including acne, psoriasis, and scleroderma, and b) compositions for transdermal delivery of MTX for treating various conditions as described, including autoimmune disorders and cancers.

2. Background Information

Drug transport through the skin occurs mainly through transcellular and intercellular pathways. However, there is increasing evidence that the transfollicular pathway plays an important role in the skin penetration of drugs, and more importantly, for the penetration of particulate systems. Although the follicular openings are less than 0.1% of the total skin surface area, these openings can be as high as 10% of the total skin area of the face and scalp.

The pilosebaceous unit is the therapeutic target for acne, seborrhetic eczema, androgenic alopecia, alopecia areata, folliculitis, and some skin cancers. Most of these conditions occur mainly in the face and/or scalp, therefore, transfollicular delivery becomes of greater importance in the development of targeted therapies against those diseases. The pilosebaceous unit is a complex and dynamic structure that includes the hair follicle, hair shaft, associated sebaceous gland and adjoining arrector pili muscle. The hair follicle is an invagination of the epidermis extending deep into the dermis, and thus, provides much greater actual area for potential penetration.

Various biochemical and immunological processes dictate the cyclic phrases of hair growth and sebum secretion. The sebaceous glad is located in the hair follicle and produces sebum, a lipid mixture of short chain fatty acids. The lower region of the hair follicle is highly proliferative and is responsibly for follicular reconstitution. The diameter of the hair follicle generally varies between 10 to 70 μm, and is known to change based on skin condition (e.g., in acne) and/or position (e.g., diameters decrease progressively toward the frontal, occipital, vertex, and parietal scalp sites).

Transfollicular delivery is influenced by the physiochemical properties of the drug, as well as the vehicle and particle size of the carrier. In addition, the hair follicle density and hair growth cycle also influence transfollicular transport. Given that the outward flow of sebum poses a physical barrier, vehicles have to be miscible with the sebum to be transported into the hair follicles.

Acne vulgaris (acne) is a multifactorial disease of the pilosebaceus unit resulting from abnormalities in sebum production, epithelial desquamation, bacterial proliferation and inflammation. Acne is one of the most common skin disorders and affects more than 60 million Americans. Around 85% of all teenagers between the ages of 12 extending to 24 suffer from acne. It is estimated that 25% of men and 50% of women will be affected by acne at some point in their adult life. Currently, topical and systemic retinoids, antimicrobial agents and hormonal agents are used to treat acne. Retinoids, which include retinol and retinoic acid, reduce acne by altering the oil chemistry on the skin. However, retinol suffers from diverse skin activity, high skin irritation, is sticky and yellow colored, water insoluble and highly photolabile, shows relatively low skin penetration/retention, and does not target particular skin sites (e.g., follicular targeting).

Given its broad anti-acne activity, tretinoin (also known as all-trans retinoic acid) is the first-line of treatment in most types of non-inflammatory and inflammatory acnes. Tretinoin induces comedolysis and normalizes the maturation of follicular epithelium to prevent future comedone formation. It also prevents inflammatory lesions by loosening follicular impactions and clearing the retained keratin from the follicular canal. In addition, it also acts as an anti-inflammatory agent by down regulating TLR-2 expression. However, the clinical efficacy of current tretinoin products on the market is sub-optimal due to the inability to achieve sufficient drug concentration at the target site in the sebaceous glands. In addition, tretinoin has multiple delivery challenges including poor water solubility, chemical instability, limited skin retention, and most importantly, strong skin irritation. Skin irritation is a major issue for patient compliance with tretinoin therapy. The skin retention of tretinoin is not only important for improving the efficacy but also for reducing systemic absorption given its teratogenic effects.

A controlled release microsphere gel formulation (0.04%) of tretinoin (RETIN-A-MICRO™, Valeant Dermatology) is available which claims improved stability and reduced skin irritation; however, the documented clinical studies show that the skin irritation of this formulation is about the same as 0.025% tretinoin. Other carriers, including lipid nanoparticles, liposomes and inorganic nanoparticles, have been studied in the literature. These approaches also have limitations in terms of drug loading, stability, multiple formulation components, and their inability to address all of the delivery challenges associated with tretinoin.

Although liposomes are widely studied delivery vehicles, they have limited drug loading capacity for hydrophobic drug such as tretinoin, in addition to poor physical stability, and limited sustained release characteristics.

Benzoyl peroxide (BPO) is another anti-acne agent. It works as a peeling agent, increases skin turnover, clearing pores and reducing the bacterial count (specifically P. acnes), as well as acting directly as an antimicrobial. However, the compound exhibits poor chemical stability, poor skin penetration and its use can lead to skin irritation, where the latter restricts administration at its most efficacious therapeutic concentration in many patients (i.e., limited to about 2.5%, compared to about 5-10%, where it is most effective).

Psoriasis is a chronic multifactorial inflammatory skin disease that affects 1-2% of the US population. Although the exact etiology of the disease is not known, it is generally considered to be an autoimmune disease where the stimulation of the immune system leads to hyperproliferation of keratinocytes at the basal layer of the epidermis and the inflammation associated with the psoriasis lesion. Abound 7.5 million Americans are affected by psoriasis with 150,000 to 260,000 cases disposed every year mainly between the ages of 15 and 35. Over half of these patients receive inadequate treatment since the current therapies are ineffective, unavailable, or unsatisfactory.

Among the several forms, psoriasis vulgaris or plaque psoriasis is the most common affecting 80% of individuals and is characterized by red raised skin (plaques) and silvery white scales in the skin. The severity of the disease varies from mild (<3% of body), moderate (3-10% of body) to severe (>10% of body) depending on the percent of the total body area affected by psoriasis. Majority (75-80%) of the patients suffer from mild to moderate psoriasis. Psoriasis has a significant effect on the quality of life of patients including causing severe depression. Patients with psoriasis also have a higher risk for developing other chronic and serious health conditions including heart disease, inflammatory bowel disease, and diabetes. The overall economic impact of the disease is estimated to be $3.2 billion annually.

Topical treatments are usually the first line of treatment for psoriasis to slow down or normalize excessive cell proliferation and reduce inflammation. Topical agents including Vitamin D analogues, corticosteroids, retinoids, or phototherapy are used for mild psoriasis, while patients with moderate to severe psoriasis are treated with systemic agents including methotrexate and immunosuppressants or biological agents. Most of these treatment options are associated with severe side effects such as toxicity from systemic agents or carcinogenicity associated with phototherapy. For majority (70-80%) of patients, topical therapy is the preferred treatment of choice. However, the current topical agents are sub-optimal due to poor skin penetration and severe side effects associated with their use (e.g., skin thinning and skin irritation). Given these challenges, there is a strong unmet clinical need to develop a safe and effective topical therapy for psoriasis to achieve a high local drug concentration in the skin and eliminate the side effects associated with systemic/topical therapy.

Methotrexate (MTX), a folic acid analogue is an anti-proliferative and anti-inflammatory agent. It inhibits DNA synthesis by irreversibly blocking the action of dihydrofolate reductase. It is an effective treatment for psoriasis and is currently administered by oral routine or injection. However, its use by physicians is limited due to the severe side effects including bone marrow toxicity, decreased white blood cell and platelet counts, liver damage, diarrhea, gastric irritation, and ulcerative stomatitis. Given that MTX has an inhibitory effect on epidermal mitosis, topical application would be an ideal treatment option for psoriasis. However, the attempts to develop a topical MTX formulation for psoriasis have met with limited clinical success mainly due to the lack of achieving sufficient drug concentration in the skin for an adequate period of time. The skin penetration of MTX is limited by its relative high molecular weight (454 Da), hydrophilicity (Log P −1.8), and its negative charge (pka=4.7) at physiological pH. Various approaches have been investigated to improve the skin penetration of MTX including use of chemical enhancers, physical methods such as iontophoresis and lipid carriers. These approaches have achieved limited success, however, due to skin irritation issues, low drug loading, and limited skin penetration. One of the key challenges is to improve the drug penetration through the altered epidermis in a psoriatic lesion, which is very different from the epidermis of normal skin. In psoriasis, the excessive stacking of parakeratotic scales leads to thickening of stratum corneum (SC) to about 10 times greater than normal skin. On the other hand the less compact SC and the disruption of the epidermal desmosomal contacts in the intercellular space in psoriatic skin can lead to increased penetration of molecules. However, the transport of delivery carriers through the altered skin barrier in psoriasis is not known.

Scleroderma is chronic disease with unknown cause and is manifested by excess synthesis and deposition of collagen in skin and connective tissues, vascular abnormalities and autoimmunity. The number of people affected with scleroderma is estimated to be between 40,000 to 165,000. Systemic scleroderma that involves skin and other major organs, while, localized scleroderma (LS) is limited to skin and subcutaneous tissues. Although uncommon, LS occurs approximately 20 times more often than systemic scleroderma, particularly in children. The estimated prevalence of LS is 50 per 100,000 before the age of 18 yrs and 220 per 100,000 by the age of 80 yrs. LS lesions in skin begin as areas of inflammation with edema, increased vascularity followed by sclerosis, collagen formation and eventual atrophy. Depending upon the location and size of the sclerodermic lesions, it can vary from superficial defects to severe functional impairment with contractures, muscle/bone atrophy and/or limb growth arrest. LS can manifest in different forms including i) morphea which is a one or more patches of sclerotic skin in one or two anatomical sites, ii) linear morphea, a more common disease in children is characterize by visible linear bands of sclerotic skin lesions in affected limb, iii) generalized morphea which is characterized by confluent plaques or multiple plaques affecting more than three sites in the body. The unknown etiology and heterogeneous nature of this rare disease coupled to the diverse diagnostic features pose a significant in the treatment of scleroderma.

There is no clear consensus on the best treatment for scleroderma, but is often dictated by the severity, extent, localization and progression of sclerotic lesions. Treatment options include topical and/or systemic agents that are directed towards suppressing inflammation, autoimmune response, collagen production and fibrosis. These include corticosteroids, vitamin D analogues (e.g. calcioptriol), ciclosporin, imiquimoid, D-penicillamine, methotrexate (MTX) and photodynamic therapy. Although there are no well-established studies on the use of corticosteroids, they are the mainstay of topical treatment in LS. Systemic treatment is used in extensive or progressive and linear forms of LS and in cases with a high risk of functional impairment. Among systemic treatments for LS, the best evidence exists for MTX alone or in combination with corticosteroids. However these treatment options have achieved limited success due to poor efficacy, problematic side effects and poor patient compliance. Therefore there is a strong need to develop an effective and safe therapeutic strategy for LS.

Several clinical studies have proven the effectiveness of systemic MTX therapy in patients with sclerotic skin disease and especially those with localized scleroderma responded well to MTX therapy. In a clinical study of 51% of scleroderma patients after one course of systemic MTX treatment (15-25 mg/weekly for 6 months) and 73% of patients treated with systemic MTX and corticosteroid reached a remission status with a median follow-up time of 55 and 58 months respectively. Patients who showed a relapse also responded to a second and third course of treatment with MTX. Further systemic MTX also was also effective in improving LS lesions in patients that failed to respond with other therapies. As a result, MTX alone or in combination with corticosteroids are currently the first line systemic treatment option for LS.

The mechanism by which MTX improves skin fibrosis is poorly understood. MTX has been shown to decrease the levels of soluble IL-2 receptors as well as the levels of IL-2, 6 and 8 on scleroderma patients. Further, the improvement in sclerotic lesions in skin with systemic MTX therapy has been attributed to its direct action on skin fibroblasts and its anti-inflammatory effects in the skin. The maximal anti-inflammatory response to systemic MTX therapy is generally delayed until several months in sclerotic skin disease which necessitate combining MTX with corticosteroids for rapid therapeutic effects. This may be possibly due to the time delay in achieving sufficient MTX concentrations in the skin for its anti-inflammatory action. However, there are no reported studies on topical MTX therapy for localized scleroderma. A major limitation of systemic MTX therapy is the severe side effects associated with its use which includes bone marrow toxicity, decreased white blood cell and platelet counts, liver damage, diarrhea, gastric irritation, and ulcerative stomatitis.

The liver damage is a major concern with long-term MTX therapy and hence requires frequent monitoring of liver function in patients. Further, the systemic MTX therapy is also limited by drug interactions. Given that LS is a self-limiting disease mainly confined to skin, topical MTX therapy can lead to a safe and effective therapy. However, the skin penetration of MTX is limited by its hydrophilicity (Log P −1.8), and its negative charge (pKa=4.7) at physiological pH. Various approaches have been investigated to improve the general skin penetration of MTX including use of chemical enhancers, lipid carriers and physical methods such as iontophoresis. These approaches have achieved limited success due to skin irritation issues, low drug loading, or limited skin penetration. One of the key challenges is to achieve optimal drug penetration/retention is sclerotic lesions, which has a thickened skin barrier compared to normal skin. Further it is important to maintain therapeutic concentrations in the sclerotic lesion to maximize the anti-proliferative and anti-inflammatory effects of MTX. Hence, there is a need to develop suitable carriers for addressing the challenges with topical delivery of MTX.

Also, when a drug is given by oral or injection there is greater exposure to liver than when given transdermally. Transdermal delivery by-passes the liver and limits the drug exposure to liver. Thus, the transdermal MTX delivery can minimize the liver toxicity of MTX.

Taken together, what is needed are carrier systems and penetration enhancer combinations that can be used to more effectively treat disorders by topical and transdermal administration.

### SUMMARY OF THE INVENTION

The present invention relates to methods and compositions for treating diverse dermatological conditions using zein-nanocarrier devices containing various drug payloads, where the zein-nanocarrier devices include shell-core nanoparticles, nanomicelles and nanoemulsions, which devices may exploit follicular/pilosebaceous unit targeting of said drug payloads for more efficacious treatment of various conditions relative to compositions that do not contain such devices. In addition, the disclosure relates to transdermal compositions for delivery of MTX systemically.

In embodiments, a composition is disclosed including a zein shell-core nanoparticle, zein-nanoemulsion or zein-nanomicelle containing a medicant, where the medicament is retinoic acid, benzoyl peroxide (BPO) or methotrexate (MTX). In one aspect, the zein core-shell nanoparticle further includes a phospholipid and a surfactant. In a related aspect, the phospholipid is lecithin and the surfactant is a block co-polymer. In a further related aspect, the block co-polymer is a poloxamer.

In another aspect, the zein-micelle includes a polyethylene glycol (PEG). In a related aspect, the zein-nanoemulsion further includes a phospholipid and a triterpene. In a further related aspect, the phospholipid is lecithin and the triterpene is squalene or squalene monohydroperoxide (Sq-OOH), and the zein-nanoemulsion exhibits a zeta-potential greater than about ±50. In another related aspect, the ratio of zein to medicament is between about 1:3.7 to about 13.5:1.

In one aspect, the zein shell-core nanoparticle, zein-nanoemulsion or zein-nanomicelle containing the medicament is formulated as a gel or a cream, and optionally contains one or more compounds selected from salicylic acid, sulfur, erythromycin or clindamycin, and adapalene.

In another embodiment, a method of treating a dermatological condition in a subject in need thereof is disclosed including administrating a cream or gel composition containing a zein shell-core nanoparticle, zin-nanoemulsion or zein-namomicelle and a medicament selected from retinoic acid, benzoyl peroxide or methotrexate (MTX).

In one aspect, the dermatological condition includes acne, seborrhetic eczema, androgenic alopecia, alopecia areata, folliculitis, hyperplastic lesions, psoriasis vulgaris, guttate psoriasis, inverse psoriasis, pustular psoriasis, and erythrodermic psoriasis, and wherein the hyperplastic lesions are selected from the group consisting of basaloid follicular hamartoma, basaloid epidermal proliferation, overlying dermal mesenchymal lesions, trichofolliculoma, sebaceous trichofolliculoma, folliculosebaceous cystic hamartoma, trichodiscoma/fibrofolliculoma, pilar sheath acanthoma, sebaceous hyperplasia, nevus sebaceous of Jadassohn, trichofolliculoma, desmoplastic trichoepithelioma, trichoblastoma, trichoblastic fibroma, trichoadenoma, proliferating trichilemmal cyst/pilar tumor, trichilemmoma, desmoplastic trichilemmoma, pilomatricoma/proliferative pilomatricoma, sebaceous adenoma, sebaceoma/sebaceous epithelioma, trichilemmal carcinoma, trochoblastic carcinoma, malignant proliferating trichilemmal cyst, pilomatrix carcinoma, sebaceous gland carcinoma, basal cell carcinoma with sebaceous differentiation, skin adnexal tumors, and scleroderma.

In another aspect, the zein core-shell nanoparticle further includes a phospholipid and a surfactant, where the nanoparticle partitions the medicament into a pilosebaceous unit.

In one aspect, the zein-micelle includes a polyethylene glycol (PEG), where the zein-micelle partitions the medicament into a pilosebaceous unit.

In another aspect the zein-nanoemulsion further includes a phospholipid and a triterpene, where the nanoemulsion partitions the medicament into a pilosebaceous unit.

In one embodiment, a formulation is disclosed including a zein-nanoemulsion containing a phospholipid, a squalene or squalene monohydroperoxide (Sq-OOH), and a medicament selected from retinoic acid, benzoyl peroxide or methotrexate (MTX), where the zein-nanoemulsion exhibits a zeta-potential greater than about about ±50.

In one aspect, the formulation is a cream. In another aspect, the formulation is a gel.

In another embodiment, a method of transdermally delivering methotrexate (MTX) to treat a condition in a subject in need thereof is disclosed including administrating a composition comprising MTX in combination with two or more solvents selected from ethanol, transcutol, isopropyl myristate (IPM), migloyl, phosphate buffer (4.0), santalol, eucalyptol, propylene glycol (PG), ethyl acetate, and combinations thereof.

In one aspect, the MTX is combined with ethanol, PG and eucalyptol. In a related aspect, the ethanol:PG:eucalyptol is present at a ratio of 5:2.5:2.5.

In another aspect, the MTX is combined with ethanol, PG and santalol. In a related aspect, the ethanol:PG:santolol is present at a ratio of 5:4:1.

In one aspect, the MTX is combined with ethanol, PG and ethyl acetate. In a related aspect, the ethanol:PG:ethyl acetate is present at a ratio of 3:1:1.

In a further related aspect, the disorder is selected from scleroderma, rheumatoid arthritis, psoriatic arthritis, lupus, sarcoidosis, Crohn's disease, vasculitis, multiple sclerosis, uterine cancer, lung cancer, head and neck cancer, osteosarcoma, trophoblastic neoplasms, and leukemia, and the administration achieves a plasma concentration of the medicament in the subject that is equivalent to a therapeutic plasma concentration of the medicament delivered via oral or injection route.

In another embodiment, a composition is disclosed including methotrexate (MTX) in combination with two or more solvents selected from ethanol, transcutol, IPM, migloyl, phosphate buffer (4.0), santalol, eucalyptol, propylene glycol (PG), ethyl acetate, and combinations thereof.

In one embodiments, the use of a zein shell-core nanoparticle, zein-nanoemulsion or zein-nanomicelle containing a medicament for the preparation of a composition for treating a dermatological condition is disclosed, where the medicament is selected from retinoic acid, benzoyl peroxide (BPO) or methotrexate (MTX).

In another embodiment, a method of enhancing transdermal permeation of methotrexate (MTX) is disclosed including combining MTX with two or more solvents selected from ethanol, transcutol, IPM, migloyl, phosphate buffer (4.0), santalol, eucalyptol, propylene glycol (PG), ethyl acetate, and combinations thereof; applying a plurality of separate MTX/solvent combinations to separate skin samples, where the MTX/solvent combinations have different solvent to solvent ratios; and determining the flux of the plurality of combinations in μg/cm²/hr across the separate skin samples, where permeation is determined to be enhanced when the flux for a combination is greater than that observed for free MTX and/or greater than that observed when the two or more solvents are ethanol and PG. p In one aspect, transdermal permeation is enhanced when MTX is combined with ethanol, PG and eucalyptol. In another aspect, transdermal permeation is enhanced when MTX is combined with ethanol, PG and santalol. In another aspect, transdermal permeation is enhanced when MTX is combined with ethanol, PG and ethyl acetate.

In a related aspect, a combination further includes the addition of ³H-MTX.

In one embodiment, a composition is disclosed including MTX and two or more solvents selected from ethanol, transcutol, IPM, migloyl, phosphate buffer (4.0), santalol, eucalyptol, propylene glycol (PG), ethyl acetate, for the treatment of a disorder selected from scleroderma, rheumatoid arthritis, psoriatic arthritis, lupus, sarcoidosis, Crohn's disease, vasculitis, multiple sclerosis, uterine cancer, lung cancer, head and neck cancer, osteosarcoma, trophoblastic neoplasms, and leukemia, where administration of the composition achieves a plasma concentration of the medicament in a subject that is equivalent to therapeutic plasma concentrations of the medicament delivered via oral or injection route.

In another embodiment, the use of a composition including methotrexate (MTX) in combination with two or more solvents selected from ethanol, IPM, migloyl, phosphate buffer (4.0), santalol, eucalyptol, propylene glycol (PG), ethyl acetate, and combinations thereof is disclosed for the preparation of a medicament for treating a disorder selected from scleroderma, rheumatoid arthritis, psoriatic arthritis, lupus, sarcoidosis, Crohn's disease, vasculitis, multiple sclerosis, uterine cancer, lung cancer, head and neck cancer, osteosarcoma, trophoblastic neoplasms, and leukemia, where when the composition is administered to a subject in need thereof, the medicament achieves a plasma concentration in said subject that is equivalent to therapeutic plasma concentrations of the medicament delivered via oral or injection route.

### BRIEF DESCRIPTION OF THE DRAWINGS

FIG. 1 shows a schematic representation of core-shell nanoparticles.

FIG. 2 shows in vitro skin penetration of retinol solution, micelles encapsulating retinol and nanoparticles encapsulating retinol through porcine skin. The skin was treated for 6 hrs and the amount of retinol in the skin and permeated across the skin at the end of 48 hrs was determined by radiochemical analysis using ³H retinol. Values are mean±SD (n=4). Significant difference at p<0.05.

FIG. 3 shows in vitro skin penetration of retinol solution, micelles encapsulating retinol and nanoparticles encapsulating retinol through porcine skin. The skin was treated for 48 hrs and the amount of retinol in the skin and permeated across the skin at the end of 48 hrs was determined by radiochemical analysis using ³H retinol. Values are mean±SD (n=4). Significant difference at p<0.05.

FIG. 4 shows in vitro skin retention of retinol solution, micelles encapsulating retinol and nanoparticles encapsulating retinol through porcine skin. The skin was treated for 6 hrs and the amount of retinol in the skin and permeated across the skin at the end of 48 hrs was determined by radiochemical analysis using ³H retinol. Values are mean±SD (n=4). Significant difference at p<0.05.

FIG. 5 shows in vitro skin retention of retinol solution, micelles encapsulating retinol and nanoparticles encapsulating retinol through porcine skin. The skin was treated for 48 hrs and the amount of retinol in the skin and permeated across the skin at the end of 48 hrs was determined by radiochemical analysis using ³H retinol. Values are mean±SD (n=4). Significant difference at p<0.05.

FIG. 6 shows a schematic set up of a sandwich model for in vitro penetration analysis.

FIG. 7 shows in vitro skin penetration of retinol from nanoparticles, the formulation was applied for 48 hrs and the amount of retinol in the skin and receptor compartment was determined by radiochemical analysis using ³H retinol.

FIG. 8 shows in vitro skin penetration of retinol from micelles, the formulation was applied for 48 hrs and the amount of retinol in the skin and receptor compartment was determined by radiochemical analysis using ³H retinol.

FIG. 9 shows confocal XZ and XYZ scan images (0-100 μm depth) of porcine skin after 6 hrs treatment with FITC labeled zein nanoparticles.

FIG. 10 shows transepidermal water loss (TEWL) after applying the formulation on SKH-1 hairless mice was measured one a day for 6 days. Values are mean±SD (n=4). Significant difference at p<0.05.

FIG. 11 shows in vitro release of retinoic acid from nanoparticles. Retinoic acid content was analyzed using UV spectrophotometer.

FIG. 12 shows in vitro release of retinoic acid from micelles. Retinoic acid content was analyzed using a UV spectrophotometer.

FIG. 13 shows an atomic force microscopy image of zein nanoemulsion:topography (left), amplitude (middle) and phase (right) views.

FIG. 14 shows in vitro skin penetration of retinoic acid solution, liposomes, nanoparticles and micelles through porcine skin. The formulation was treated for 48 hrs and the amount of retinoic acid in the receptor compartment was determined by radiochemical analysis using ³H retinoic acid. Values are mean±SD (n=4). Significant difference at p<0.05.

FIG. 15 shows retinoic acid release from the marketed and in-house formulations. Values are mean±SD (n=4).

FIG. 16 shows distribution of retinoic acid in various areas of hamster ear (in vitro) at the end of 24 hrs treatment.

FIG. 17 shows distribution of retinoic acid in stratum corneum, epidermis/dermis and receptor compartment at the end of 24 hrs treatment in hamster flank skin. Values are mean±SD (n=3). Significant difference at p<0.05.

FIG. 18 shows in vitro skin penetration of benzoyl peroxide formulations through full-thickness porcine skin. The formulation (equivalent to 100 μg of benzoyl peroxide) was applied for 48 hrs and the amount of drug in the skin and receptor compartment was determined by radiochemical analysis using ³H-BPO (0.25 μCi). Values are mean±SD (n=3), Significant difference at p<0.05.

FIG. 19 shows in vitro release of MTX from zein-lecithin-PLURONIC F68 nanoparticles (circle), zein-nanoemulsion (square) and liposomes (triangle) in phosphate buffer (pH 7.4). The drug concentration was determined by UV-spectrophotometry at 300 nm.

FIG. 20 shows in vitro skin penetration of methotrexate solution, liposomes and nanoparticles through porcine full-thickness skin. The formulation was treated for 6 hrs and the amount of methotrexate in the skin at the end of 6 hrs was determined by radiochemical analysis using ³H-MTX. Values are mean±SD (n=3). Significant difference at p<0.05.

FIG. 21 shows in vitro skin penetration of MTX formulations through full-thickness porcine skin. The formulation (equivalent to 100 μg of MTX) was applied for 48 hrs and the amount of MTX in the skin and receptor compartment was determined by radiochemical analysis using ³H-MTX (0.025 μCi). Values are mean±SD (n=3), Significant difference at p<0.05.

FIG. 22 shows in vitro skin penetration of methotrexate solution, liposomes and nanoparticles through human skin. The formulation was treated for 48 hrs and the amount of methotrexate in the skin and permeated across the skin at the end of 48 hrs was determined by radiochemical analysis using ³H-MTX. Values are mean±SD (n=4). Significant difference at p<0.05.

FIG. 23 shows in vitro skin disposition of methotrexate solution, liposomes and nanoparticles through porcine full-thickness skin. The formulations were treated for 6 hrs and then removed. The amount of methotrexate in the receptor medium was determined for different time periods. At the end of 48 hrs, the amount of methotrexate in skin and receptor was determined by radiochemical analysis using ³H-MTX. Values are mean±SD (n=3), Significant difference at p<0.5.

FIG. 24 shows in vitro skin penetration of methotrexate solutions through porcine full-thickness skin. The formulation was treated for 6 hrs and the amount of methotrexate in the stratum corneum and epidermis/dermis at the end of 6 hrs was determined by radiochemical analysis using ³H-MTX. Values are mean±SD (n=3).

FIG. 25 shows in vitro skin penetration of methotrexate solutions through porcine (PS) and human (HS) full-thickness skin. The formulation was treated for 6 hrs and the amount of methotrexate in the stratum corneum and epidermis/dermis at the end of 6 hrs was determined by radiochemical analysis using ³H-MTX. Values are mean±SD (n=3). Significant difference at p<0.05.

FIG. 26 shows in vitro skin penetration of methotrexate solutions and formulations through porcine full-thickness skin. The formulation was treated for 48 hrs and the amount of methotrexate in the receptor compartment at various time points was determined by radiochemical analysis using ³H-MTX. Values are mean±SD (n=3). Significant difference at p<0.05.

FIG. 27 shows in vitro skin penetration of methotrexate solutions and formulations through porcine full-thickness skin. The formulation was treated for 48 hrs and the amount of methotrexate in the skin at the end of 48 hrs was determined by radiochemical analysis using ³H-MTX. Values are mean±SD (n=3). Significant difference at p<0.05.

FIG. 28 shows in vitro skin penetration of methotrexate solutions and nanoparticle dispersion through full thickness psoriatic and non-psoriatic human skin. The formulation was treated for 48 hrs and the amount of methotrexate in the skin at the end of 48 hrs was determined by radiochemical analysis using ³H-MTX. Values are mean±SD (n=3). Significant difference at p<0.05.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nanoparticle” includes one or more nanoparticles, and/or compositions of the type described herein which will become apparent to those persons skilled in the art upon reading this disclosure and so forth.

Unless defined otherwise, all technical and scientific terms used herein have the same meaning as commonly understood one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of particular term and “substantially” and “significantly” will mean plus or minus >10% of the particular term.

Zeta potential is a scientific term for electrokinetic potential in colloidal systems. When the potential is low, attraction exceeds repulsion and the dispersion will break and flocculate. So, colloids with high zeta potential (negative or positive) are electrically stabilized, which colloids with low zeta potentials tend to coagulate or flocculate. For example, values from 0 to ±5, rapid coagulation or flocculation is expected; from ±10 to ±30, incipient instability is expected; from ±30 to ±40, moderate stability is expected; from ±40 to ±60, good instability is expected; and more than ±61, excellent stability is expected.

As used herein, “nanoemulsion” means a mixture of one or more liquids with a prolamine where the prolamine is immiscible (i.e., nonmixable or unbendable) in the one or more liquids, and which mixture forms a dispersion, where the size of the particles formed in the dispersion are in the nanometer range (i.e., between about 1-999 nm). For example, as disclosed herein, a nanoemulsion may be formed by combining zein (a prolamine) in ethanol and water to form a hydroalcoholic solution, which combination is then added to an aqueous solution containing lipids (e.g., squalene and lecithin) to form a dispersion, where the particles in the dispersion exhibit sizes in the range of about 149 to about 160 nm.

The present zein-nanocarriers (i.e., nanoparticles, nanoemulsions and nanomicelles) and penetration enhanced MTX compositions as disclosed may be used to treat skin conditions, including but not limited to, acne, seborrhetic eczema, androgenic alopecia, alopecia areata, folliculitis, hyperplastic lesions, psoriasis vulgaris, guttate psoriasis, inverse psoriasis, pustular psoriasis, and erythrodermic psoriasis, psoriatic arthritis, basaloid follicular hamartoma, basaloid epidermal proliferation, overlying dermal mesenchymal lesions, trichofolliculoma, sebaceous trichofolliculoma, folliculosebaceous cystic hamartoma, trichodiscoma/fibrofolliculoma, pilar sheath acanthoma, sebaceous hyperplasia, nevus sebaceous of Jadassohn, trichofolliculoma, desmoplastic trichoepithelioma, trichoblastoma, trichoblastic fibroma, trichoadenoma, proliferating trichilemmal cyst/pilar tumor, trichilemmoma, desmoplastic trichilemmoma, pilomatricoma/proliferative pilomatricoma, sebaceous adenoma, sebaceoma/sebaceous epithelioma, trichilemmal carcinoma, trichoblastic carcinoma, malignant proliferating trichilemmal cyst, pilomatrix carcinoma, sebaceous gland carcinoma, basal cell carcinoma with sebaceous differentiation, and skin adnexal tumors, scleroderma, rheumatoid arthritis, psoriatic arthritis, lupus, sarcoidosis, Crohn's disease, vasculitis, multiple sclerosis, uterine cancer, lung cancer, head and neck cancer, osteosarcoma, trophoblastic neoplasms, and leukemia.

The present disclosure relates to the following compounds:

i) Prolamines, which are proline rich plant storage proteins (e.g., zein, gliadin, and the like), where zein is a biodegradable FDA approved prolamine which has similar characteristics to skin keratin and is therefore a skin compatible nanocarrier. In embodiments, zein nanoparticles, nanoemulsions, and nanomicelles produce a water-washable formulation of retinoic acid.

ii) Triterpenes, are terpenes consisting of six isoprene units and have the molecular formula C₃₀H₄₈ (e.g., squalene or related compounds), and are lipids that may be found in sebum secretions, including that in embodiments, formulations may use such triterpenes (e.g., in nanoemulsions or protein-lipid nanoparticles), thus resulting in a sebum compatible formulation.

iii) Sebum—a target site for acne treatment.

iv) Retinol (Formula I):

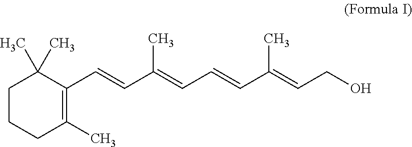 [SMILES: CC1=C(/C=C/C(C)=C/C=C/C(C)=C/CO)C(C)(C)CCC1]

Molecular formula: C₂₀H₃₀O

Molar mass: 286.45 g/mol

Melting point: 62-64° C.

Appearance: Yellow to orange with a brown cast powder

Solubility in water: nearly insoluble

Solubility in fat: soluble

v) Retinoic acid (Formula II):

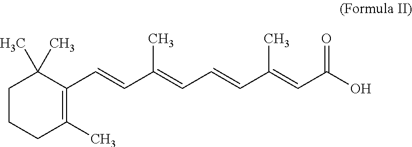 [SMILES: CC1=C(/C=C/C(C)=C/C=C/C(C)=C/C(=O)O)C(C)(C)CCC1]

Molecular formula: C₂₀H₂₈O₂

Molar mass: 300.43512 g/mol

Appearance: yellow to light orange crystalline powder with characteristic floral odor

Melting point: 180-182° C., crystals from ethanol

Solubility in water: nearly insoluble

Solubility in fat: soluble

v) Methotrexate (Formula III):

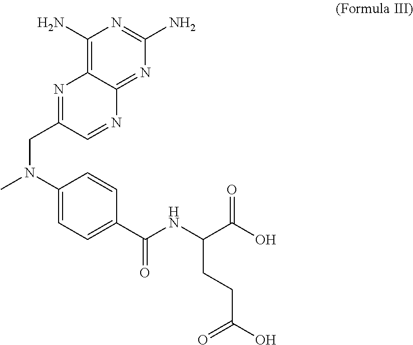 [SMILES: CN(Cc1cnc2nc(N)nc(N)c2n1)c1ccc(C(=O)NC(CCC(=O)O)C(=O)O)cc1]

Molecular formula: C₂₀H₂₂N₈O₅

Molecular Mass: 454.4 g/mol

Appearance: Yellow powder

Melting point: 195° C.

Solubility in water: <0.1 g/100 ml at 19° C.

Solubility in DMSO: 100 mM.

vi) Benzoyl Peroxide (Formula IV)

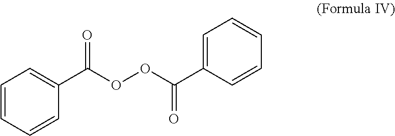 [SMILES: O=C(OOC(=O)c1ccccc1)c1ccccc1]

Molecular formula: (C₆H₅O)₂O₂

Molar mass: 242.0676 g/mol

Appearance: white powder

Melting point: 104-106° C.

Solubility in water: insoluble

Soluble in ether and chloroform

vii) Surfactants, such as poloxamers, which are nonionic triblock copolymers composed of a central hydrophobic chain of polyoxypropylene (poly(propylene oxide)) flanked by two hydrophilic chains of polyoxyethylene (poly(ethylene oxide)), and which are commercially known as PLURONICS®, SYNPERONICS®, and KOLLIPHOR®.

vii) Phospholipids, comprising at least a diglyceride, and a phosphate group. In embodiments, such a phospholipid is lecithin.

Polyethylene glycol (PEG, PEO, or POE) refers to an oligomer or polymer of ethylene oxide.

As disclosed herein, nanocarriers show high encapsulation efficiency for retinoic acid, including nanocarrier formulations as disclosed which produce a free flowing and non-hygroscopic powder of retinoic acid.

In embodiments, the release of retinoic acid is sustained from nanoparticles, nanoemulsions, and nanomicelles. In a related aspect, submicroscopic nanomicelles comprise a plant protein and a second FDA-approved polymer that form self-assembled micelles with high encapsulation efficiency to enhance the delivery of water-insoluble molecules. In a further related aspect, nanocarriers as disclosed result in higher skin retention and reduced systemic absorption of retinoic acid which leads to enhanced efficacy and safety relative to free retinoic acid formulations. Further, the nanocarrier formulations as disclosed achieve higher retinoic acid delivery into hair follicles and sebaceous glands, which are target sites for acne, versus free retinoic acid formulations.

Other advantages include that the encapsulation overcomes the skin irritation of free retinoic acid; the nanoformulations significantly improve the chemical stability of retinoic acid; the lyophilized zein nanocarriers may be easily incorporated into various topical formulation matrices such as gel, creams, lotions or ointments; and generally water washable formulation may be used for cosmetic and dermatological applications.

Retinoic acid is a water insoluble hydrophobic compound. The encapsulated retinoic acid in zein nanoparticles/nanoemulsions/nanomicelles is water soluble/dispersible. Hence nanoparticles/nanoemulsions/nanomicelles may be used to develop water washable retinoic acid formulations for topical applications.

The stability and shelf life of retinoic acid is limited by its poor stability. Encapsulation of retinoic acid in nanoparticles, nanoemulsions and nanomicelles may significantly enhance the stability and shelf-life of retinoic acid formulations. Retinoic acid release may be sustained from zein nanoparticles, naonoemulsions and nanomicelles. Release may be sustained from 2-10 days depending on whether nanoparticles, nanoemulsions or nanomicelles are used. This reduces the dose and frequency of application of retinoic acid.

Retinoic acid has poor skin penetration properties. Nanoparticles, nanoemulsions and nanomicelles lead to enhanced skin penetration of retinoic acid. Retinoic acid may be retained in the top or lower layers of the skin using nanoparticles, nanoemulsions and nanomicelles for various dermatological/cosmetic applications.

Retinoic acid topical application is mainly associated with the severe skin irritation and it is a major non-compliance for the dermatological applications. As disclosed herein, topical application of encapsulated retinoic acid in nanoparticles, nanoemulsions and nanomicelles will significantly reduce the skin irritation caused by retinoic acid.

Other retinoids may also be encapsulated in zein nanoparticles/nanoemulsions/nanomicelles for cosmetic and dermatological applications. Apart from treatment of acne, retinoic acid is widely used for various skin disorder products such as in sunscreens, anti-psoriatic, anti-acne and skin-cancer products along with other drugs. Further, other agents such as anti-oxidants, free-radical scavengers, anti-inflammatory agents may also be encapsulated along with retinoic acid in nanoparticles/nanoemulsions/nanomicelles as disclosed.

In embodiments, core-shell nanoparticles (100-300 nm) using zein, a hydrophobic biodegradable protein from corn as the core and lecithin (a natural phospholipid) and PLURONIC F68 (amphiphilic, non-ionic polymeric surfactant) as the shell, is disclosed. The technology may be used to encapsulate a wide range of drug molecules with high encapsulation efficiency. Zein nanoparticles as disclosed herein are non-immunogenic and show sustained release for more than 10 days in vitro. The amphophilic characteristics of these core-shell nanoparticles may be used to enhance the skin penetration/retention of both hydrophilic and hydrophobic drug molecules. In embodiments, this technology may be applied for the targeted delivery of therapeutic agents to hair follicles.

In embodiments, zein nanoparticles show 4 fold higher drug encapsulation and two fold higher skin penetration than liposomes. In embodiments, cosmetic anti-aging retinoid encapsulated in zein nanoparticles lead to higher skin retention with minimal systemic exposure, in addition to significantly improving its chemical stability, skin retention and sustained release. In a related aspect, skin irritation may be completely prevented after encapsulation of retinoids in zein nanoparticles. In another related aspect, retinol nanoparticles were found to mainly localize within hair follicles. In embodiments, nanoparticles, nanomicelles, and nanoemulsions may be combined to target various strata in the skin, such that different distributions of drug to, e.g., the stratum corneum, ventral dermis, pilosebaceous glands, cartilage and dorsal dermis, may be achieved to enhance efficacy. In a related aspect, in view of partitioning sebum effects, nanoemulsions>nanomicelles>nanoparticles. However, while not being bound by theory, nanomicelles may show higher skin deposition. In a further related aspect, it will be apparent to one of skill in the art that by altering the ratio of zein/lecithin/squalene in the nanoemulsions, sebum partitioning may be further enhanced.

As disclosed herein, zein nanoparticles of sufficient particle size have been developed for follicular targeting of tretinoin for treatment of acne. In a related aspect, the nanoparticles as disclosed herein may be used to develop topical formulations (e.g., lotion, gel or cream). In a further related aspect, the efficacy of said formulations has been tested in an acne animal model (e.g., Rhino mouse model).

Although there are a numerous tretinoin products in the market, none of these products are designed for targeted delivery to the follicles. One of the main advantages of the formulations as disclosed is the development of a delivery system for targeting the hair follicles, including the use of natural biodegradable skin compatible materials. While not being bound by theory, the present formulations take advantage of the fact that zein has similar characteristics to skin keratin and lecithin has similar characteristics to skin lipids. Similarly, PLURONIC F68 is an amphiphilic polymer that may interact with both the hydrophilic and hydrophobic domains in the skin. Further, the carrier formulations as disclosed are compatible with sebum resulting in better targeting to the pilosebaceous unit (e.g., see above for nanoemulsions). Given the high sebum secretion in acne, it is important for the carrier to be compatible with sebum for achieving high drug concentration in the sebaceous glands (e.g., zein-nanoemulsions). Current formulations are available in concentration from 0.01 to 0.1% of tretinoin. Some of these products use a higher concentration of tretinoin to overcome the poor chemical stability, but the higher concentration also increases skin irritation.

The microsphere encapsulated gel formulation of tretinoin (RETIN-A-MICRO™) is the only approved encapsulated formulation on the market with only marginal benefit over unencapsulated tretinoin formulations. While not being bound by theory, the use of a natural, biodegradable plant biopolymer to generate nanoparticles for skin drug delivery technology platform allows for facile penetration through the hydrophilic and hydrophobic domains of the skin. For example, in the case of zein-nanoemulsions, the components therein mimic the skin protein and lipids present in said skin domains.

The tretinoin nanoparticle formulations as disclosed herein offer significant advantages over existing products in terms of improved chemical stability, higher skin retention, controlled release and reduced skin irritation. While not being bound by theory, the composition and size of the zein nanoparticles afford more specific targeting of tretinoin toward the acne site in hair follicles.

Additional advantages of zein nanoparticles over other polymeric nanoparticles are as follows: i) zein is a biodegradable protein from corn with a long history of safe use in food and pharmaceutical industries; ii) zein nanoparticles may achieve a high encapsulation efficiency for both hydrophilic and hydrophobic molecules; iii) since zein is a hydrophobic protein, it may sustain the drug release for prolonged periods; iv) multiple amino acids in zein provides the flexibility of modifying the surface properties of said nanoparticles such as by cross-linking, co-polymerization among others moieties which may be used to optimize particle size, drug encapsulation and drug release. In embodiments, all the materials used in the nanoparticles may be GRAS (generally regarded as safe) materials.

In embodiments, the zein nanoparticles may be optimized for drug loading, particle size, drug release and stability via skin penetration of the nanoparticles in vitro using human skin to maximize follicular targeting by varying the particle size, drug amount and treatment time. In a related aspect, the skin irritation and follicular/pilosebaceous targeting of nanoparticles may be tested in vivo in mice and hamsters, respectively. Microencapuslated tretinoin (RETIN-A-MICRO™) may be used for comparison.

A core-shell nanoparticle (100-200 nm) has been generated using zein, a hydrophobic biodegradable protein from corn as the core, lecithin (a natural phospholipid) and PLURONIC F68 (amphiphilic, non-ionic polymeric surfactant) as the shell, which combination is used to encapsulate a wide range of drug molecules with high encapsulation efficiency and may sustain the drug release for prolonged periods. Zein nanoparticles as disclosed herein are non-immunogenic and do not show any skin irritation. The amphiphilic characteristics of these core-shell nanoparticles may be used to enhance the skin penetration/retention of both hydrophilic and hydrophobic drug molecules.

In embodiments, tretinoin nanoparticle formulation may be modified by altering the drug amount and drug/zein ratio to obtain nanoparticles with optimal size, drug loading, release and stability. Release of tretinoin may be determined using diffident buffers (pH 7.4 and 4.5) to simulate the skin conditions, including that artificial sebum may be used. The photostability of tretinoin nanoparticles may be determined by exposing the formulation to UV light.

In embodiments, tretinoin nanoparticles may be prepared by phase separation method as disclosed in US Pub. No. 2012/0195947, herein incorporated by reference in its entirety. Tretinoin and zein may be dissolved in 90% ethanol. The hydroalcoholic solution may be added under probe sonication to the buffer solution (citrate buffer, pH 7.4) containing PLURONIC F68 and lecithin as stabilizers followed by evaporation of alcohol by mechanical stirring for about 2 to 3 hours. The nanoparticles may be separated by centrifugal filtration followed by lyophilization. Amounts of tretinoin and drug/zein/lecithin/PLURONIC F68 ratio may be varied to optimize the particle size and drug loading. The size and zeta potential of the nanoparticles may be measured using dynamic light scattering (Nicomp ZLS 380). The morphology and size homogeneity may be measured by atomic force microscopy. The encapsulation efficiency of tretinoin may be determined by dispersing the tretinoin nanoparticles in 90% ethanol and then assaying the tretinoin concentration in a UV spectrophotometer at 320 nm. The encapsulation efficiency may be calculated from the ratio of amount of tretinoin loaded to the total tretinoin added and the amount of tretinoin with respect to the amount of zein may be used to calculate the loading efficiency.

The release of tretinoin from zein nanoparticles may be modulated between pH 4.5 and 7.4 by placing the nanoparticles in a dialysis bag at 37° C. The sink conditions may be maintained by using 10% alcohol in the release medium. To determine the drug release in sebum, artificial human sebum may be used. The artificial human sebum is composed of triglycerides, wax esters, squalene, cholesterol and cholesterol esters. The ingredients may be heated at 60° C. with stirring until all the solids become a clear liquid. The nanoparticles may be incubated with the artificial sebum at 37° C. At various time points aliquots may be sampled and centrifuged. The supernatant may be dilated with 90% alcohol and analyzed by high performance liquid chromatography (HPLC). In a related aspect, a C18 column and a mobile phase consisting of 50.6:24.4:25.6% (v/v) of acetonitrile/methanol/2.5% aqueous ammonium acetate may be used. Tretinoin may be detected at 340 nm.

Both solid-state and liquid state stability of tretinoin nanoparticles may be analyzed by storing at 4° C. and at 25° C. in a stability chamber for 1 month. For liquid state stability analysis, the tretinoin nanoparticles may be dispersed in pH 7.4, while lyophilized powder may be used for solid-state stability analysis. To test the photostability, the formulations may be exposed to UVA radiation (320-400 nm) using an UV lamp at 30 J/cm² for 4 hours. Samples may be removed periodically to determine the drug content by HPLC.

Zein nanoparticles of different size may be obtained by varying the formulation factors. In embodiments, nanoparticles in the size range of 100-300 nm and high drug loading for follicular targeting are disclosed. In embodiments, the encapsulation in the nanoparticles improves tretinoin stability. To ensure that tretinoin is stable during preparation, the exposure to light may be limited and if required anti-oxidants may be included in the formulation. Encapsulation of tretinoin in hydrophobic zein may result in sustained release. The release may be further tuned by cross-linking zein nanoparticles.

In embodiments, zein nanoparticles target tretinoin to hair follicles and achieve therapeutic concentrations more efficaciously compared to free tretinoin and marketed tretinoin formulations (e.g., specific targeting of follicles, increase sustained release, increased penetration, and the like). Further, skin retention of tretinoin may be improved and systemic absorption may be reduced. Human facial skin may be used for in vitro skin penetration analysis.

In selecting optimal particle size for follicular targeting, FITC labeled zein may be used. FITC may be conjugated to amino groups in zein using EDC as a catalyst. The conjugate may be purified by dialysis and characterized by NMR. Nanoparticles prepared using FITC-zein (in PBS pH 7.4) may be used for skin penetration studies as described below. Skin may be cryosectioned and imaged by fluorescence microscopy. Free FITC may be used for comparison.

The dermatomed human skin (400 μm) may be thawed to room temperature and sandwiched between the two compartments of a vertical Franz diffusion cell (0.64 cm² diffusion area, Permegear, USA). The receptor compartment may be maintained at 37° C. with stirring. Sink conditions may be maintained by adding 10% ethanol. To ensure that the skin is intact, transepidermal water loss (TEWL) may be measured before starting the experiment using a vapometer (Delfin, Sweden). 200 μl of different doses of free or encapsulated tretinoin formulation in PBS (pH 7.4) may be applied to the donor compartment for 6-48 hrs. At the end of treatment, the skin surface may be washed and blotted dry. The skin area exposed to the treatment may be cut and the SC may be removed by tape-stripping (15-20 tape strips) using pre-weighed scotch tapes. First 1-2 tape strips may be discarded to remove the surface adsorbed drug.

To quantify drug in the hair follicles, after tape stripping a drop of cyanoacrylate glue may be placed on the skin and the glue may be covered with a glass slide under slight pressure. After 5 minutes, the polymerized cyanoacrylate and the glass slide may be removed with one quick movement. The remaining skin (epidermis+dermis) may be minced. Receptor sample may also be removed for analysis. Tretinoin from the tape strip, scrapping from the follicular cast and the skin samples may be extracted with 90% ethanol. The extract may be centrifuged and the supernatant may be analyzed by HPLC. To determine whether the nanoparticles form a depot in the skin, in a separate set of experiments, the skin may be treated with free and encapsulated formulation and after 6 hrs the skin may be cleaned with 10% ethanol and replaced with plain buffer. The samples from the receptor may be analyzed for 6-48 hrs. After 48 hrs the tretinoin in SC, follicular casts and epidermis/dermis may be analyzed as described herein. Results may be reported as percent of applied dose, amount/cm² of the skin and amount/gm of the tissue. All the experiments may be done in quadruplicate and may be repeated with two skins from different donors. The results may be compared using one-way ANOVA at p<0.05.

A sandwich epidermis model may be used to analyze follicular delivery. For example, epidermis may be removed from human skin by immersing the skin for 45 seconds in a water bath at 60° C. The epidermis may then be gently teased off the underlying dermis and floated on a 0.002% w/v sodium azide solution. Epidermal membrane may be floated with inner side down in a 0.0001% trypsin solution with 0.5% w/v sodium hydrogen carbonate maintained at 37° C. After 12 hrs, the SC membrane may be picked up on a filter paper and washed off with water. Subsequently, SC/epidermal sandwich may be prepared by placing SC membrane on top of epidermal membrane derived from adjacent skin region. This sandwich membrane may be used for skin penetration analysis. At the end of 48 hrs, the amount of cargo molecule (e.g., tretinoin) in epidermis/dermis and receptor compartment may be determined by HPLC. The results may be compared with the skin penetration of cargo molecule through epidermis. The percent follicular contribution may be calculated using the formula [1-[2(Q_{sand}/Q_{cp})] 100 where Q_{sand} and Q_{cp} are the amounts of cargo molecule in receptor medium for sandwich epidermis and single epidermis, respectively.

While not being bound by theory, the amount of tretinoin in follicles may be relatively less compared to the amount in skin. However, the nanoparticles are expected to show a higher follicular targeting than free and control microsphere formulation. In one aspect, rubbing the formulation on the skin (consistent with application in humans) may help in increasing follicular penetration of nanoparticles. In embodiments, different sized nanoparticles (100-300 nm) may be used to maximize the follicular targeting of tretinoin. In embodiments, therapeutically relevant concentrations are achievable by altering the applied dose and treatment time.

In embodiments, skin irritation and follicular/pilosebaceous targeting of tretinoin nanoparticles may be carried out in vivo using animals. Skin irritation may be carried out in hairless mice, while the follicular targeting may be analyzed using hairy Balb/c mice and hairless mice. Hamster ear model may be used to study the targeting of tretinoin nanoparticles to sebaceous glands.

SKH-1 hairless mice (6-7 weeks old) may be procured from Charles River Laboratories (Wilmington, Mass.). Free (in 10% ethanolic solution) or encapsulated tretinoin (in phosphate buffer pH 7.4) formulation (0.5% w/v) may be applied on a 1 cm² of dorsal skin by gentle rubbing. The formulation may be applied once daily for 7 days. The transepidermal water loss (TEWL) may be measured every day before treatment. The animals may also be observed for signs of skin irritation such as erythema, skin dryness and peeling by three independent observers and may be scored on a scale from 0-5 (no erythema to severe skin erythema). At the end of the analysis, the mice are sacrificed and the skin cryosectioned to observe for gross histological changes. Sodium lauryl sulfate (0.1% w/v) may be used as a positive control, while a no treatment group serves as the negative control. Results may be compared using one-way ANOVA at p<0.05.

In embodiments, age-matched hairy Balb-c mice may be used. Hairless mice do not have well developed and functional hair follicles unlike the hairy Balb-c mice. The dorsal hair on the Balb-c mice may be removed using a hair-clipper the day before the experiment while avoiding cuts to the skin. FITC (in 10% ethanolic solution) or FITC labeled zein nanoparticles (in phosphate buffer pH 7.4) formulation (0.5% w/v) may be applied on 5 cm² of dorsal skin by gentle rubbing under isoflurane anesthesia. After the treatment period (0, 3 and 24 hrs) the residual formulation may be cleaned using 10% ethanol. The mice may be sacrificed by CO₂ asphyxiation. The treated skin area may be cut and placed in a cryomold followed by covering with OCT (optimum cutting temperature). The mold may be kept in −70° C. freezer. The tissue may be sectioned (5 μm thick) in a cryotome and may be fixed in 5% formalin and then mounted in aqua mount for fluorescent visualization using fluoroscence microscope with a FITC filter. Three animals may be used for each treatment.

Syrian male hamsters of at least 12 weeks of age may be purchase from Charles River Laboratories (Wilmington, Mass.) The ventral middle ear of the hamster has a high density of pilosebaceous unit that is very similar in number and size in humans. More specifically, they are comparable to the sebaceous glands in the face and scalp in humans where acne usually occurs. Hamster may be anesthetized with isoflurane anesthesia and around 20-50 μl of the formulation may be applied and carefully spread over the central portion of the ventral ear surface. Both the ears may be used for treatment and one ear from three different animals may be used for each formulation. At the end of 6 hours, the animals may be sacrificed and the ears may be dissected to isolate the sebaceous glands. The formulation may be removed using a KIMWIPE™ and the surface may be tape stripped about 25 times. The ventral and dorsal surface may be separated and the sebaceous glands may be gently scrapped from the ventral dermis. Complete removal of the sebaceous gland may be confirmed using light microscope. The drug may be extracted using 90% ethanol, centrifuged and the supernatant may be analyzed by HPLC. The amount of tretinoin may be represented as μg/cm² and percent of applied dose. The results may be compared using one-way ANOVA at p<0.05.

In embodiments, tretinoin nanoparticles as disclosed show less skin irritation than free tretinoin or commercial formulations. While not being bound by theory, since the hamster model resembles the pilosebaceous units in the scalp and face in humans and given that it is the target site for acne, the results provide a good estimate for clinical translation. In embodiments, the tretinoin formulation may be incorporated in a gel formulation or the solution may be applied multiple times. In a related aspect, the dose may vary from 0.1% to about 0.1% w/w. One of skill in the art would appreciate that the dose and/or treatment time may be altered as necessary.

In other embodiments, BPO may be used in combined with nanocarriers as disclosed. For example, nanoparticles may be prepared by dissolving zein and BPO (50 mg) ethanol (about 90%). The hydroalcoholic solution may then be combined with lecithin and PLURONIC F68 dispersed in citrate buffer (pH 7.4), which combination may be sonicated (10 min, 37% amplitude) with 10 sec on and 1 sec off cycle. The solution may be kept under stirring at room temperature for about 2-3 hrs. The entrapped and free BPO may be separated using centrifugal filters, and the BPO nanoparticles dispersion may be stored under refrigerator until use.

Other nanocarrier embodiments for BPO may include zein micelles and zein nanoemulsions.

In addition, BPO-nanocarriers may combine salicylic acid, sulfur, erythromycin or clindamycin (antibiotics), and adapalene (a synthetic retinoid). In a related aspect, common combination drugs include benzoyl peroxide/clindamycin and adapalene/benzoyl peroxide.

Benzoyl peroxide for acne treatment is typically applied to the affected areas in gel or cream form, in concentrations of about 2.5% increasing through about 5%, and up to about 10%. 5% and 10% concentrations are slightly more effective than 2.5%, while 2.5% is usually better tolerated. As disclosed herein, because the zein nanocarriers reduce skin irritation, application of higher effective concentrations of BPO is made possible.

Other common uses for benzoyl peroxide include dyeing hair, and as an active ingredient in teeth whitening systems.

The typical concentration for benzoyl peroxide is about 2.5% to about 10% for both prescription and over-the-counter drug preparations that are used in treatment for acne. Higher concentrations are used for hair bleach and teeth whitening.

In embodiments, micelles may be prepared from PEG-zein dispersed in ethanol (90%), where the dispersion may be incubated for about 10 hrs at about 37° C. under stirring. The dispersion is then dialyzed against water for about 6-8 hrs by changing the water about 6-8 times. The micelle dispersion may be lyophilized, and the lyophilized micelles stored under refrigeration (e.g., about 3 to about 5° C.).

In embodiments, nanoemulsions may be prepared by mixing zein (about 10-14 mg, in embodiments about 13.5 mg) in ethanol followed by dissolving in water (0.2 ml), where the mixture is dispersed and sonicated for about 20 seconds. The resulting mixture is added drop wise to a solution containing about 15 ml of citrate buffer pH (7.4), squalene (about 0.5%=0.075 ml), and lecithin (about 50-65 mg) and sonicated for about 5 min, with 10 sec on and 1 sec off cycle, about 37% amplitude at about 10° C. (ice bath). The alcohol is evaporated under magnetic stirring at room temperature (about 2-3 hrs). The emulsion is then subjected to centrifugal filtration (MW about 10000 Da), at about 4000 rpm for about 60 minutes, about 4° C. The nanoemulsion may be stored at room temperature.

In embodiments, nanoemulsions comprising benzoyl peroxide may be made by mixing zein (about 25 mg) and BPO (about 50 mg) in ethanol (90%), where the mixture is combined with lecithin (about 65 mg) and squalene (about 0.075 ml), which lipids may be dispersed in citrate buffer (about 15 ml, pH 7.4), by drop wise addition of the ethanol solution to the citrate buffer solution under probe sonication (10 min, 37% amplitude), with 10 sec on and 1 sec off cycle. Further, the resulting solution may be kept under stirring at room temperature for about 3 hrs. The entrapped and free benzoyl peroxide may be separated by using centrifugal filters, and the benzoyl peroxide dispersion may be stored in a refrigerator until use. In a similar fashion, BPO nanoparticles maybe prepared by incorporating 10 μl of hot ¹⁴C BPO along with 50 mg of BPO.

In embodiments, zein nanoemulsions show high drug encapsulation compared to zein-lecithin-PLURONIC nanoparticles and PEG-zein micelles. Further, the size of the zein nanoemulsions was smaller than the zein-lecithin-PLURONIC nanoparticles. In a related aspect, zein nanoemulsions result in 2 fold increase in skin penetration of BPO compared to zein-lecithin-PLURONIC nanoparticles and 3 fold higher skin penetration than BPO solution. The encapsulation may also improve the chemical stability and reduce the skin irritation of BPO.

As disclosed herein, nanocarriers show high encapsulation efficiency for MTX, including that zein nanoparticles showed a 3 fold higher encapsulation of MTX than liposomes. Unlike the liposomes, the MTX release from the nanocarriers was sustained in vitro for a week. Further, the skin penetration of MTX in nanocarriers was enhanced by 3 fold compared to a simple aqueous solution and 2 fold compared to liposomes in excised porcine skin. Moreover, a 2.5 fold reduction in transport across the skin into the receptor phase compared to simple MTX solution was observed. In embodiments, MTX nanoparticle formulations are disclosed that are efficacious skin penetrators, to include demonstrating efficacy in a human psoriatic skin. In a related aspect, topical formulations are disclosed (e.g., lotion, gel or cream) where efficacy and topical bioavailability in a pre-clinical human psoriatic skin transplanted mouse model, may be demonstrated.

Zein has similar characteristics to skin keratin, while lecithin, a skin penetration Enhancer, has characteristics to skin lipids. PLURONIC F68 is an amphophilic polymer that can interact with both the hydrophilic and hydrophobic domains in the skin. While not being bound by theory, the high keratin and high free fatty acid content in psoriatic lesions, the amphiphilic core-shell zein nanoparticles may interact with the psoriatic lesion to improve the skin penetration of MTX. In embodiments, the MTX nanoparticles afford significant advantages in terms of improved skin penetration, higher skin retention, and sustained release compared liposomes or free MTX.

Advantages of the formulations as disclosed herein include i) that zein is a biodegradable protein from corn with a long history of safe use in food and pharmaceutical industries, ii) that the formulations may achieve high encapsulation efficiencies for MTX, iii) since zein is a hydrophobic protein, it can sustain the drug release for prolonged periods, and iv) multiple amino acids in zein provide the flexibility of modifying the surface properties such as cross-linking, co-polymerization, and the like, to optimize particle size, drug encapsulation, and drug release.

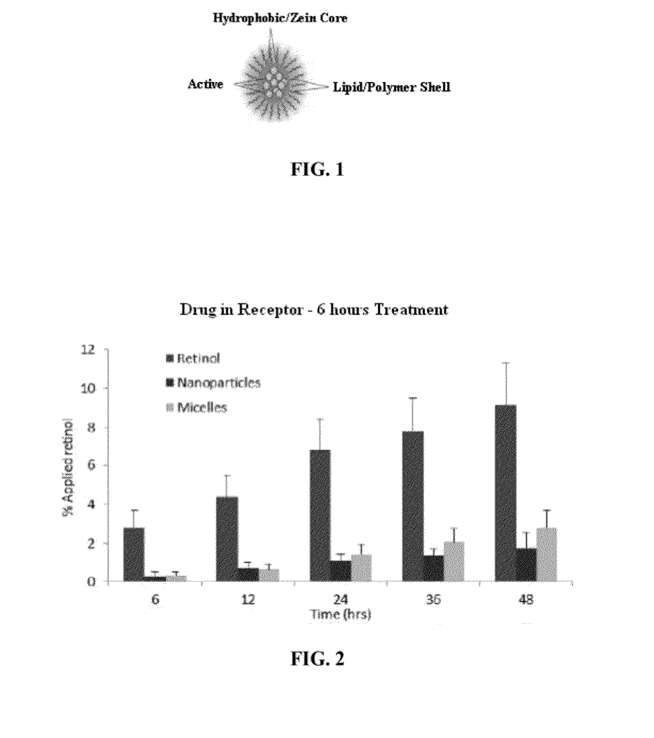

In embodiments, core-shell nanoparticles may be prepared using zein as the core with lecithin and PLURONIC F68 as the shell. The nanoparticles may be prepared by phase separation method based on the differential solubility of zein in hydroalcoholic solution (90% alcohol) and pH 7.4 buffer. In one aspect, zein may be dissolved in hydroalcoholic solution, while lecithin and PLURONIC F68 may be dissolved in pH 7.4 buffer. The addition of the hydroalcoholic phase followed by evaporation of ethanol forms core-shell nanoparticles (FIG 1). The nanoparticles are spherical and have core-shell architectures. The particle sizes of the nanoparticles vary from about 90-170 nm with a low polydispersity index (about 0.1-0.3). Molecules varying in Log P from −0.5 to 6.2 may be encapsulated in the zein nanoparticles. Molecules of varying molecular weight from 100-4000 Da may be encapsulated in zein nanoparticles with high encapsulation efficiency (60-96%). The particle size and encapsulation efficiency may be optimized by varying the formulation parameters. In a related aspect these, results as disclosed herein demonstrate the broad applicability of zein nanoparticles as a delivery vehicle.

In embodiments, fluoroisothiocaynate (FITC), a hydrophobic dye and rhodamine, a hydrophilic dye may be encapsulated in zein nanoparticles. In one aspect, skin penetration may be analyzed using excised porcine skin. Free or nanoparticle encapsulated dyes (in PBS, pH 7.4) may be applied to the skin in a vertical skin diffusion cell for 6 hrs or 48 hrs. The skin may then be washed with PBS, snap-frozen in liquid nitrogen and cryo-sectioned (7 μm). The section may be observed under fluorescence microscope. The free dye was mainly localized to the stratum corneum (SC), while the dye in nanoparticles was found in the viable epidermis and dermis. While not being bound by theory, the amphiphilic characteristics of core-shell nanoparticles enables the encapsulated molecule to partition into both lipophilic and hydrophilic domains and reach the epidermis and dermis which are the target sites for most skin diseases including psoriasis.

In one aspect, zein nanoparticles loaded with MTX were compared with liposomes loaded with MTX. As disclosed herein, although the particle size was comparable, the nanoparticles showed a lower polydispersity index (PI) compared to liposomes. As disclosed herein, the encapsulation efficiency was 3 fold higher with zein nanoparticles compared to liposomes. In one aspect, the in vitro release of MTX from zein nanoparticles was tested in phosphate buffer (pH 7.4) and was sustained up to a week. In comparing in-vitro skin penetration of free MTX and encapsulated MTX, both liposomes and nanoparticles increased the skin penetration of MTX and reduced the transport of drug across the skin into the receptor medium. However, zein nanoparticles showed 2 fold higher skin penetration than liposomes. As disclosed herein, the MTX concentration achieved in porcine skin with nanoparticles is close to the therapeutic plasma concentrations reported for humans (i.e., 4-8 μg or between about 0.01 to 0.1 μg/ml. By optimizing parameters, therapeutic concentrations may be achieved in human skin.

In embodiments, optimizing MTX nanoparticle formulations may be accomplished by altering the drug amount and drug/zein ratio to obtain nanoparticles with optimal size, drug loading, release and stability. In embodiments, release of MTX may be analyzed in different buffers (pH 7.4 and 4.5) to simulate skin conditions. The storage stability of MTX nanoparticles may be tested to check for particle aggregation and MTX stability.

In embodiments, MTX nanoparticles may be prepared using the phase separation method as disclosed in US Pub. No. 2012/0195947, herein incorporated by reference in its entirety. MTX and zein may be dissolved in 90% ethanol. The hydroalcoholic solution may be added under probe sonication to the buffer solution (citrate buffer, pH 7.4) containing PLURONIC F68 and lecithin as stabilizers followed by evaporation of alcohol by mechanical stirring for 3 hours. The nanoparticles may be separated by centrifugal filtration followed by lyophilization. The amount of MTX and drug/zein/lecithin/PLURONIC F68 ratio may be varied to optimize the particle size and drug loading. The size and zeta potential of the nanoparticles may be measured using dynamic light scattering (Nicomp ZLS 380) and size homogeneity may be measured by atomic force microscopy.

In other embodiments, MTX-nanoemulsions are disclosed, and such nanoemulsions may be prepared by dissolving MTX and zein in a hydroalcoholic solution (e.g., 90% ethanol). The hydroalcoholic solution may be added to citrate buffer (pH 7.4) containing squalene and lecithin under probe sonication and stirred in a magnetic stirrer for 2-3 hrs to remove alcohol. Free MTX may be separated from encapsulated MTX using centrifugal filter (MWCO: about 10 kDa).

The encapsulation efficiency of MTX may be determined by dispersing the MTX nanoparticles in 90% ethanol and centrifuged followed by determination of MTX concentration in the supernatant by UV spectrophotometry at 300 nm. The encapsulation efficiency may be calculated from the ratio of amount of MTX loaded to the total MTX added and the amount of MTX with respect to the amount of zein (determined using protein assay) may be used to calculate the leading efficiency.

The release of MTX from zein nanoparticles may be analyzed at pH 4.5 and 7.4 (i.e., to mimic the skin surface pH and pH in epidermis/dermis respectively) by placing the nanoparticles in a dialysis bag at 37° C. At various time points aliquots may be sampled and centrifuged. The supernatant may be analyzed by UV spectrophotometry at 300 nm.

Both solid-state and liquid stale stability of MTX nanoparticles may be analyzed by storing at 4° C. and 25° C. in the refrigerator and stability chamber respectively for 1 month. For liquid state stability analysis, the MTX nanoparticles may be dispersed in a buffer at pH 7.4, while lyophilized powder may be used for solid-state stability analysis. The particle size and MTX concentration may be determined as described above.

In embodiments, stable nanoparticles are obtained in the size range of 100-200 nm with a low polydispersity index and high drug loading for skin penetration studies. In one aspect, zein particles in the above size range may be transported into the skin through intercellular spaces and hair follicles. In addition to other formulation parameters, the pH of the aqueous phase may be altered to increase the MTX loading in nanoparticles. Encapsulation of MTX in zein nanoparticles may sustain the drug release to reduce the dosing frequency. However, a higher loading may limit the sustained release characteristics in which case the release may be sustained by cross-linking zein nanoparticles.

In embodiments, therapeutic activity and IC₅₀ may be determined to define the target concentration for skin penetration analysis. For such analysis normal and psoriatic reconstructed human skin models may be used.

Reconstructed human epidermis and psoriatic tissues are commercially available, and may be purchased from Mattek Corporation (Ashland, Mass.) and maintained in the tissue culture medium provided by the manufacturer. The tissue may be treated with different concentrations of free and nanoparticle-encapsulated MTX (about 1.5 to about 6.4×10⁻⁷ M in phosphate buffer, pH 7.4) for about 4-24 hours. Blank nanoparticles and PBS (pH 7.4) may be used as negative controls. The tissue may then be rinsed several times with serum free media to remove the test substances, and transferred to fresh medium followed by incubation for another 18 hours. The MTT assay may be performed by transferring the tissues to 24-well plates containing MTT medium (1 mg/ml). After about 3 hours, formazan salt formed by cellular mitochondria may be extracted with 2.0 ml/tissue of isopropanol and the optical density may be measured using a plate reader at 570 nm. Relative cell viability may be calculated as % of the mean of the negative control. The IC₅₀ may be estimated by the time necessary to kill 50% of the cells using Graphpad Prism. The analysis may be done in quadruplicate and the results may be compared using one-way ANOVA at p<0.05.

To measure the specific effect of MTX on keratinocyte proliferation, proliferation markers including nuclear proliferation marker Ki-67 and proliferating cell nuclear antigen (PCNA) may be measured by immunohistochemistry. The reconstructed normal and psoriatic tissue may be treated with free MTX and MTX nanoparticles at three different MTX concentrations for about 12-24 hrs. The tissues may be washed several times with the tissue culture media and may be fixed in buffered 4% formalin for 4 hours, and processed for routine histology. Sections (6 μm) may be stained with hematoxylin and eosin or processed for immunohistochemical staining using an indirect immunoperoxidase technique with avidin-biotin complex enhancement. Primary antibiotics including PCNA AB-1 mouse monoclonal antibody with a dilution of 1:50 and Ki-67 Ab-2 mouse monoclonal antibody with a dilution of 1:50 may be used. After half-an hour, the slides may be incubated in biotinylated goat anitpolyavalent sera for 10 minutes and in streptavidin peroxidase for 20 min. Finally AEC substrate system (Labvision, Fermont, Calif. USA) may be applied for about 3 min. After incubation, the sections may be rinsed with distilled water and the tissue may be counterstained with hematoxylin. The slides may be observed under an optical microscope. The level of staining may be scored on the following 4-point scale: no staining (grade 0), moderate focal/faint diffuse staining (grade 1), strong focal/moderate diffuse staining (grade 2), and strong diffuse staining (grade 3). The average scoring of three tissues may be used to compare the different treatment groups. Wilcoxon-signed rank test at p<0.05 may be used for statistical analysis.

The human reconstructed model has been used to evaluate the efficacy of anti-psoriatic drugs as it is known to express the important biomarkers of psoriasis. As opposed to the use of a cell culture model, the reconstructed human skin model is closer to the in vivo environment. Although the skin barrier integrity in reconstructed human tissue model may be lower than human skin, it may provide a reasonable estimate of the skin penetration and activity of the MTX nanoparticles. The results from this analysis may be validated in vivo in a psoriasis mouse model.

Dermatomed human skin (400 μm) may be thawed to room temperature and may be sandwiched between the two compartments of a vertical Franz diffusion cell (0.64 cm² diffusion area, Permegear, USA). The receptor compartment may be maintained at 37° C. with stirring. To ensure that the skin is intact, transepidermal water loss (TEWL) may be measured before starting the experiment using a vapometer (Delfin, Sweden). To the donor compartment, 200-400 μl of different doses of free or encapsulated MTX formulation in PBS (pH 7.4) may be applied for 6-48 hrs. At the end of treatment, the skin surface may be washed repeatedly with acetate buffer (pH 4.5):ethanol (1:1) followed by several washes with double distilled water to remove excessive MTX on the skin surface. The skin area exposed to the treatment may be cut and the SC removed by tape-stripping (15-20 tape strips) using pre-weighed scotch tapes (Transpore, 3M), The remaining skin (epidermis+dermis) may be minced. Receptor sample may also be removed for analysis. MTX from the tape strip and the skin samples may be extracted with acetate buffer (pH 4.5):ethanol (1:1) under shaking in a water bath at 25° C. for 5 hrs. The extract may be centrifuged and the supernatant may be analyzed by HPLC. To determine whether the nanoparticles form a depot in the skin, in a separate set of experiments, the skin may be treated with free and encapsulated formulations and after 6 hrs the skin may be washed as described herein and replaced with plain PBS buffer (pH 7.4). The samples from the receptor may be analyzed for 6-48 hrs. After 48 hrs the MTX in SC and epidermis/dermis may be analyzed as described herein. Results may be reported as percent of applied dose, amount per cm² of the skin and amount per gm of the tissue. All the experiments may be done in quadruplicate and may be repeated with two skins from different donors. The results may be compared using Student's t-test at p<0.05.

In the case of psoriatic skin, the uninvolved and psoriatic lesional skin may be used for skin penetration analysis using the same procedure as described herein. The receptor phase may consist of Dulbecco's modified Eagle's medium with 10% fetal bovine serum to maintain the viability of the tissue. The conditions identified to be optimal with the normal human skin may be used for this analysis and the results may be compared to normal human skin and uninvolved skin from a psoriasis patient.

The MTX concentration may be determined using a HPLC (Waters 2695, USA). The samples may be separated in a symmetry C18 column (column size 4.6 mm×150 mm) using a mobile phase consisting of acetate buffer:acetronitrile solution (90:10) at a flow rate of 1 ml/min, an injection volume of 20 μl followed by detection at a wavelength of 302 nm. The MTX concentration may be determined from the calibration curve of MTX at various concentrations.

To gain an understanding of the difference in zein nanoparticle transport through psoriatic skin and normal skin, FITC labeled zein may be used. FITC may be conjugated to amino groups in zein using EDC as a catalyst. The conjugate may be purified by dialysis and characterized by NMR. Nanoparticles prepared using FITC-zein (in PBS pH 7.4) may be used for skin penetration studies (6 hrs) as described herein. Skin may be cryosectioned and imaged by fluorescence microscopy. Free FITC may be used for comparison.

In embodiments, an advantage of this disclosure is the use of a zein-based nanocarrier for topical drug delivery to psoriatic lesions. Another advantage is the development of a topical therapy to maximize one or more of the multiple pharmacological actions of a highly effective systemic anti-psoriatic drug without the risk of systemic toxicity and frequent monitoring of patients for organ toxicity. The amphiphilic nanoparticles as disclosed herein comprise biodegradable and skin compatible materials that have similar characteristics to skin proteins and lipids. Zein has similar characteristics to skin keratin and lecithin is a skin penetration enhancer with similar characteristics to skin lipids. Similarly PLURONIC F68 is a non-ionic polymeric surfactant and skin penetration enhancer. While not being bound by theory, the core-shell nanoparticles may partition both into hydrophilic and hydrophobic domains in the skin to enhance the skin penetration and retention of MTX. Given the high keratin and free fatty acid in human psoriatic lesions, the amphiphilic core-shell nanoparticles may be an ideal carrier to improve the skin. The nanoparticles as disclosed herein showed a 3 fold higher encapsulation efficiency compared to liposomes. Further the nanoparticles as disclosed herein showed sustained the MTX release while hardly any sustained release was seen with liposomes. Moreover, the nanoparticles as disclosed herein showed at least a 2 fold higher skin penetration compared to liposomes, including that the nanoparticles showed higher MTX penetration into psoriatic lesions compared to free MTX and compared to MTX nanoparticles in normal skin.

Although psoriatic plaques are different in composition to sclerotic plaques, both the diseases are characterized by thickened skin barrier. In embodiments, MTX nanoparticles may also be used to achieve higher penetration into sclerotic plaques. Squalene is major lipid found in the sebum secreted by sebaceous glands in the skin to protect and lubricate the skin surface. In addition to improving the drug encapsulation and skin penetration of zein nanoparticles, squalene also has collagen inhibitory activity. Squalene undergoes oxidation in the skin to squalene monohydroperoxide (Sq-OOH), and may function as an anti-oxidant to protect the skin against photodamage. Sq-OOH has also been shown to reduce the collagen production on topical application in mouse. While not being bound by theory, given that the basal levels of Sq-OOH in the skin is very low, an exogenous supply of Sq-OOH may have benefit effect in reducing the collagen production in skin sclerotic lesions. In embodiments, the core-shell nanoparticles mediated topical delivery of MTX as disclosed herein offers a platform for development of a safe and efficacious therapy for localized scleroderma.

In other embodiments, a topical therapy to maximize one or more of the multiple pharmacological actions of a highly effective systemic scleroderma drug without the risk of systemic toxicity and frequent monitoring of patients for organ toxicity is disclosed. The core-shell nanoparticles may be composed of biodegradable and skin compatible materials that have similar characteristics to skin proteins and lipids.

Zein has similar characteristics to skin keratin and lecithin is a skin penetration enhancer with similar characteristics to skin lipids, while squalene is a skin sebum lipid with skin penetration enhancer activity. Taken together, the nanocarriers as disclosed herein may partition both into the protein and lipid domains in the skin to enhance the skin penetration and retention of MTX. The degradation product of squalene (Sq-OOH) has collagen inhibitory activity which can further enhance the efficacy of the MTX topical therapy in LS.

In addition, the nanoparticles as disclosed herein may provide controlled drug release to maintain MTX concentrations in the skin sclerotic lesions for effective anti-inflammatory and anti-proliferative activity.

In other embodiments, free MTX may be combined with various solvents for transdermal delivery. In a related aspect, combinations may include, ethanol:PG (1:1); ethanol:PG:santalol (5:4:1); ethanol:PG:eucalyptol (5:2.5:2.5); ethanol:PG:ethyl acetate (6:2:2). In one embodiment, combinations exhibit the following flux and penetration hierarchy:ethanol:PG:eucalyptol (5:2.5:5:2.5)>ethanol:PG:santalol (5:4:1)>ethanol:PG:ethyl acetate>ethanol:PG (1:1).

In other embodiments, MTX may be formulated with combinations of solvents, including but not limited to, ethanol:PG (A), ethanol:ethyl oleate (B), ethanol:ethyl oleate:eucalyptol (C), ethanol:ethyl oleate:santalol (D), ethanol:PG:transcutol (E), ethanol:PG:transcutol:santalol (F), ethanol:PG:transcutol:eucalyptol (G), ethanol:transcutol:ethyl oleate (H), ethanol:transcutol:ethyl oleate:eucalyptol (I). In a related aspect, the combinations (A)-(I) may be 2:1 for combinations (A) and (B); 5:2.5:2.5 for combinations (C) and (D). For combinations (E)-(I), the combinations may be from at least 2:1. to about 5:2.5:2.5, depending on the number of solvents contained therein.

The formulations of the present disclosure may be used to develop topical formulations (lotion, gel, or cream).

The following examples are intended to illustrate but not limit the invention.

### EXAMPLES

### Example 1

### General Particle Preparation

Zein Preparation Method.

Zein particles may be prepared by the method as described in US Pub. No. 2011/0091565, herein incorporated by reference in its entirety. As examples, coumarin (0.0066 g), doxirubicin (0.001 g), dextran FITC (0.003 g), 5-fluorouracil (5 mg), and pDNA (0.187 g) were added to the various dispersion to produce encapsulated particles.

In an alternative method of the invention, the ultrasonic shear of the second phase solution may be supplemented or replaced by high pressure homogenizer by passing the dispersion under high pressure through a narrow orifice for reducing the particle size. This may be especially useful to produce nanoparticles in the smaller size range when a high concentration of zein is used. Also, high pressure homogenization may be used as a scale-up method for preparing zein nanoparticles.

Liposome Preparation

Lecithin (54 mg) and cholesterol (12 mg) were dissolved in 5 ml of chloroform in a round bottom flask. The chloroform was evaporated at 40° C. using rotavapor and a thin film of lipids was formed inside the round bottom flask. The round bottom flask was stored in a dessicator overnight. The film was hydrate using 5 ml of phosphate buffer (pH 7.4) at 35° C. using rotavapor for 1 hr. The entrapped and free retinoic acid were separated using ultracentrifugation at 50000 rpm for 1 hr. The resulting pellet was re-dispersed in 1 ml of phosphate buffer (pH 7.4), sonicated for 5 minutes and stored under refrigeration.

Micelle Preparation.

PEG-zein (100 mg) was dispersed in ethanol (90%) and incubated for 10 hrs at 37° C. under stirring. Further, the dispersion was dialyzed against water for about 6-8 hrs by changing the water 6-8 times. The micelle dispersion was lyophilized, and the lyophilized micelles were stored under refrigeration.

Nanoemulsion Preparation

Zein (13.5 mg) in ethanol (2 ml) was dissolved in water (0.2 ml) and dispersed and sonicated for 20 seconds. The resulting mixture was added to drop wise to a solution containing 15 ml of citrate buffer pH (7.4), squalene (0.05%=0.075 ml), and lecithin (65 mg) and sonicated for 5 min, with 10 sec on and 1 sec off cycle, 37% amplitude at 10° C. (ice bath). The alcohol was evaporated under magnetic stirring at room temperature (2-3 hrs). The dispersion was subjected to centrifugal filtration (MW 10000 Da), at 4000 rpm for 60 minutes; 4° C. The dispersion was stored at room temperature.

Zein Core-Shell Nanoparticle Results.

In general, zein core-shell articles have an average size of between about 100 to about 300 nm, which may encapsulate a wide variety of molecules. They are non-immunogenic and biocompatible (i.e., no skin irritation). Sustained release≧10 days in vitro. Results of encapsulation may be seen in Table 1.

TABLE 1
Encapsulation data for Zein Core-Shell Particles.

| | | | | Particle Size | Encapsulation |
|---|---|---|---|---|---|
| Compound | MWt. | | Log P | (nm) | Efficiency (%) |
| Coumarin | 178 | Da | 1.2 | 173 ± 20 | 68 ± 6 |
| Doxorubicin-HCl | 580 | Da | −0.5 | 171 ± 45 | 61 ± 16 |
| Dextran FITC | 4000 | Da | −1.2 | 89 ± 12 | 79 ± 8 |
| pDNA | 3.2 | kbp | <0.5 | 185 ± 12 | 86 ± 3 |

In summary, zein shell-core nanoparticles may be used to encapsulate diverse compounds. Further, such encapsulation enhances solubility and stability of the encapsulated compound, including that such nanoparticles afford higher skin penetration and retention of the cargo molecules delivered. Moreover, the nanoparticles afford sustained release, follicular targeting and overall improved performance compared to liposomes.

Zein Micelle Results.

In general, zein micelles have an average size of between about 120 to about 200 nm, which may encapsulate a wide variety of molecules. Sustained release≧24 hours in vitro. Results of encapsulation may be seen in Table 2.

TABLE 2
Characteristics of Zein Micelles.

| | | | Particle | | |
|---|---|---|---|---|---|
| | Log | MWt. | Size | | Encapsulation |
| Compound | P | (Da) | (nm) | PI | Efficiency (%) |
| Doxorubicin | 1.2 | 543.5 | 153 ± 3 | 0.18 ± 0.06 | 92 ± 6 |
| Curcumin | 2.5 | 368.3 | 124 ± 4 | 0.25 ± 0.03 | 95 ± 4 |
| Nile Red | 5 | 318.3 | 165 ± 7 | 0.21 ± 0.08 | 77 ± 11 |
| Retinol | 6.2 | 285.5 | 191 ± 6 | 0.27 ± 0.06 | 83 ± 3.1 |

Table 3 compares the characteristics between liposomes and zein micelles.

TABLE 3
Liposome and Zein-Micelle (Curcumin formulations) Comparisons.

| | Particle Size | | Encapsulation |
|---|---|---|---|
| Formulation | (nm) | PI | Efficiency (%) |
| Liposomes | 256 ± 20 | 0.25 ± 0.03 | 65 ± 6 |
| Zein-Micelle | 124 ± 4 | 0.18 ± 0.02 | 95 ± 4 |

In summary, the micelles afford encapsulation of lipophilic compounds, including enhancing solubility and stability of such compounds. Further, the micelles increase skin penetration and retention of active compounds. Sustained release was observed, and follicular targeting was achieved with the micelles, including that the micelles showed overall improved performance compared to liposomes.

The technology summary for the zein shell-core nanoparticles, zein nanoemulsions and zein micelles is shown in Table 4.

TABLE 4
Technology Summary.

| | Zein Shell Core | Zein | Zein- |
|---|---|---|---|
| Parameters | Nanoparticles | Micelles | Nanoemulsions |
| Molecules | Diverse | Hydrophobic | Diverse |
| Dispersibility/ | ++ | +++ | ++ |
| solubility | | | |
| Sustained Release | +++ | ++ | ++ |
| (SR) | | | |
| Skin penetration | + | ++ | +++ |
| enhancement | | | |
| Skin retention | ++ | +++ | ++ |
| Follicular Targeting | ++ | ++ | ++ |
| Sebum partitioning | + | ++ | +++ |

+++ indiucates best of three, ++ next best and + next best

### Example 2

### Zein-Retinol Analysis

Zein-Retinol Nanoparticle Preparation.

Zein-retinol particles may be prepared by the method as outline in US Pub. No. 2012/0195947, herein incorporated by reference in its entirety.

Micelle Preparation.

PEG-Zein (100 mg) and retinol (1 mg) were dispersed in ethanol (90%) and incubated for 10 hrs at 37° C. under stirring. Further, the dispersion was dialyzed against water for about 6-8 hrs by changing the water 6-8 times. The micelle dispersion was lyophilized, and the lyophilized retinol containing micelles were stored under refrigeration.

Zein-Retinol Nanoparticles and Micelles Increase Water Dispersability, Stability, and Sustain Retinol Release.

The nanoparticles and micelles were free flowing and readily dispersible (colorless) in aqueous solution, while free retinol powder (yellow) was sticky and was not dispersible in water. Encapsulation of retinol into the zein nanoparticles and micelles improved the stability of retinol by 2 to 5 fold (see Table 5).

TABLE 5
Stability of Retinol Encapsulated in Nanoparticles.

| | |
|---|---|
| Light | Dark |

Half-life (hrs)

| | | |
|---|---|---|
| Liquid State | | |
| Retinol dispersion | 16.11 ± 1.9 | 20.83 ± 2.1 |
| Nanoparticle dispersion | 42.0 ± 2.5 | 94.81 ± 7.4 |
| Solid State | | |
| Retinol dispersion | 52.75 ± 3.5 | 63.0 ± 2.7 |
| Nanoparticle dispersion | 92.66 ± 8.4 | 153.0 ± 20.2 |

Porcine In-Vitro Skin Penetration Analysis (6 hrs Treatment).

Skin penetration studies were carried out using full thickness porcine skin (n=3) for 6 hrs at 37° C. under stirring using vertical Franz diffusion cell. The encapsulated retinol solution was spiked with ³H retinol, where the ³H spiked micelles and nanoparticles were dispersed in 400 μl of phosphate buffer (pH 7.4) and taken in the donor compartment. The receptor compartment was filled with 5 ml of phosphate buffer (pH 7.4).

At the end of 6 hrs, 200 μl of sample from the receptor compartment was collected and analyzed for the retinol content. Simultaneously, the amount of retinol in the skin was determined using radiochemical method of analysis. Results are shown in FIG. 2.

Porcine and Human Skin Penetration Studies (48 hrs Treatment).

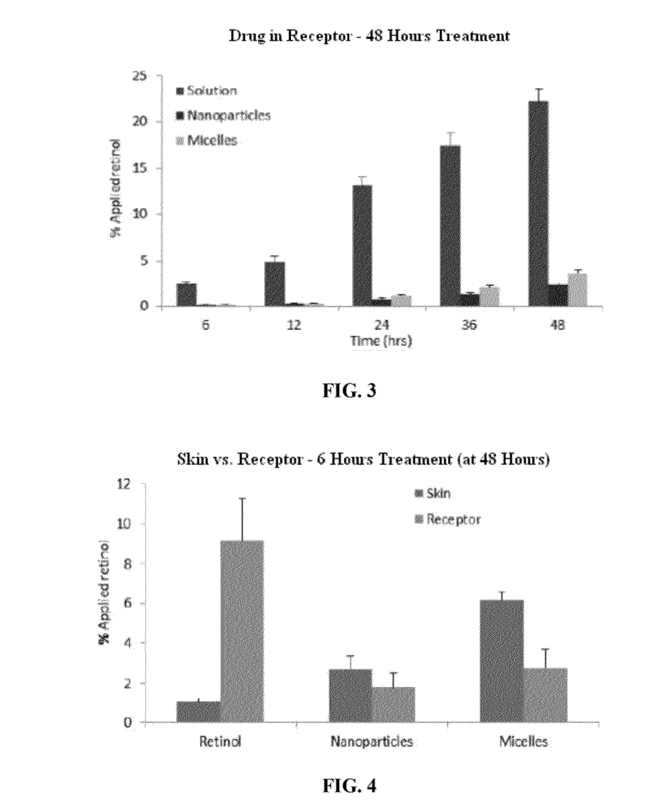

The method was similar to that for the 6 hour treatment except that at the end of 48 hours the amount of retinol was determined. Results may be seen in FIG. 3.

Zein-Retinol Nanoparticles and Micelles Increase Skin Retention, Target Follicles and Reduce Skin Irritation of Retinol.

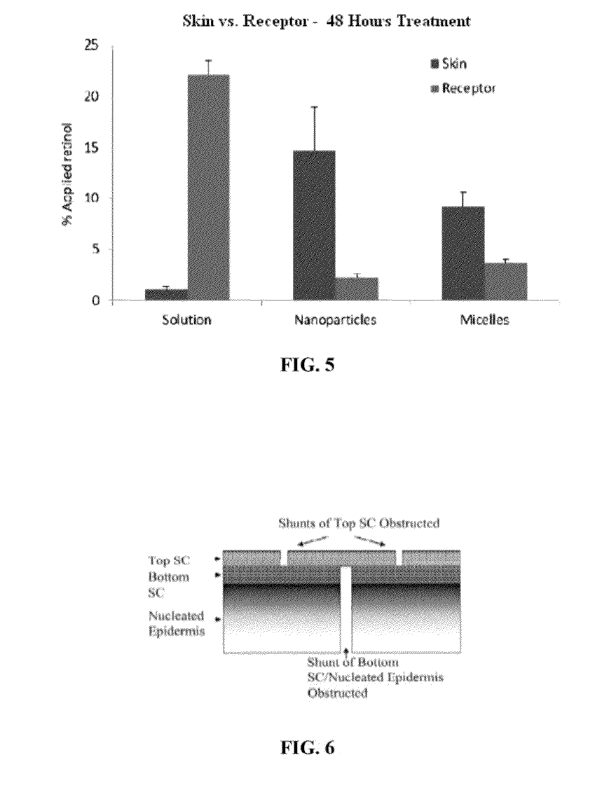

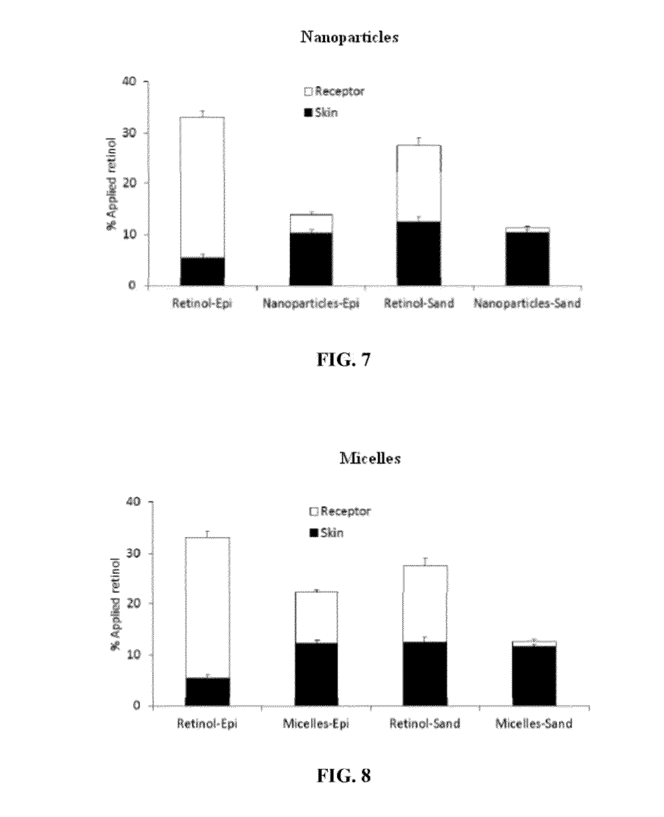

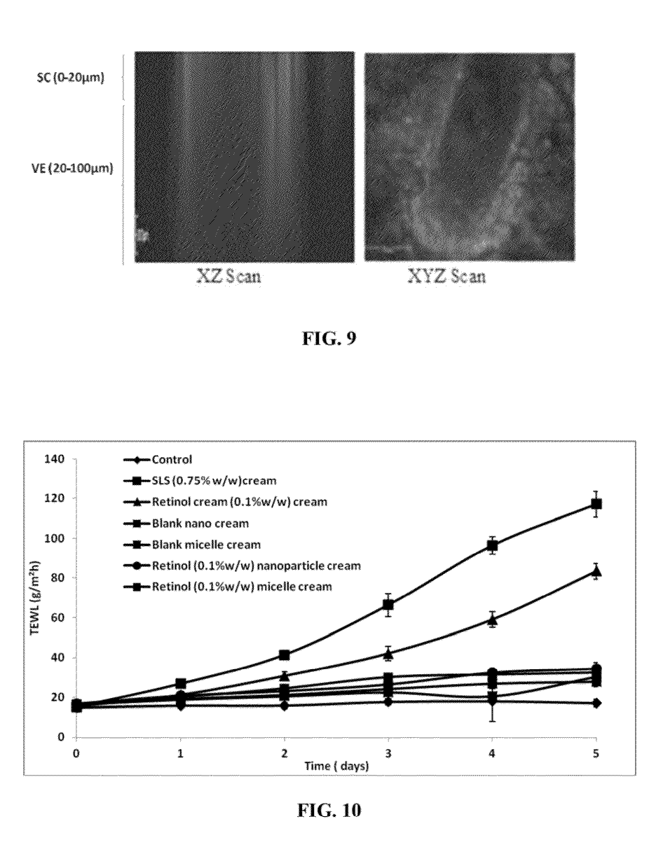

Retinol nanoparticles and micelles showed significantly higher skin retention and lower transport across the skin compared to free retinol at 6 and 48 hrs treatment (FIGS. 4 and 5, respectively). In another set of experiments, stratum corneum (SC) was removed and sandwiched over porcine epidermis followed by retinol skin penetration analysis (model shown in FIG. 6). In this case, the follicular pathways are blocked by the SC placed over the epidermis. Although the skin transport is expected to decrease because of the increase in the skin thickness, a significant reduction is attributed to the blocking of the follicular pathways. Unlike the free retinol, there was significant reduction in the transport of retinol nanoparticles (−50%) indicating that trey are transported through hair follicles (FIG. 7). Similar results were obtained with retinol micelles (FIG. 8). This was further substantiated from confocal microscopy studies where FITC labeled zein nanoparticles were mainly localized in hair follicles (FIG. 9). To test the skin irritation, free, nanoparticle encapsulated, and micelle encapsulated retinol cream formulations were applied on the back of SKH-1 hairless mice once a day for 5 days. The transepidermal water loss (TEWL) was measured every day. As can be seen from FIG. 10, the free retinol cream caused an increase in TEWL, while the nanoparticle and micelle formulations did not show any sign of skin irritation and the values were similar to negative control (i.e., no treatment). Sodium lauryl sulfate (SLS), a known skin irritant was used as the positive control. Taken together the data support the use of zein nanoparticles and micelles for follicular targeting and address other delivery challenges see with retinol.

### Example 3

### Zein-Retinoic Acid Analysis

Zein-Retinoic Acid Nanoparticle Preparation.

Zein (13.5 mg) and retinoic acid (1 mg) were dissolved in ethanol (90%), lecithin (65 mg) and PLURONIC F68 (130 mg) were dispersed in 15 ml citrate buffer (pH 7.4). Ethanol solution was added drop wise to citrate buffer solution under probe sonication, where sonication was carried out for 10 mm (37% amplitude) with 10 sec on and 1 sec off cycle. Subsequently, the solution was kept under stirring at room temperature for about 3 hrs and the entrapped and free retinoic acid were separated using centrifugal filters. The nanoparticle dispersions were lyophilized using trehalose as stabilizer, and the lyophilized retinoic acid nanoparticles were stored under refrigeration.

Zein-Retinoic Acid Micelle Preparation.

PEG-zein (100 mg) and retinoic acid (1 mg) were dispensed to ethanol (90%) and incubated for 10 hrs at 37° C. under stirring. Further, the dispersion was dialyzed against water for about 6-8 hrs by changing the water 6-8 times. The micelle dispersion was lyophilized, and the lyophilized retinoic acid micelles were stored under refrigeration.

Zein-Retinoic Acid Liposome Preparation.

Lecithin (54 mg), cholesterol (12 mg) and retinoic acid (1 mg) were dissolved in 5 ml of chloroform in a round bottom flask. The chloroform was evaporated at 40° C. using rotavapor and a thin film of lipids was formed inside the round bottom flask. The round bottom flask was stored in a desiccator overnight. The film was hydrated using 5 ml of phosphate buffer (pH 7.4) at 35° C. using rotavapor for 1 hr. The entrapped and free retinoic acid were separated using ultracentrifugation at 50000 rpm for 1 hr. The resulting pellet was re-dispersed in 1 ml of phosphate buffer (pH 7.4), sonicated for 5 minutes and stored under refrigeration.

Similarly the nanoparticles, micelles and liposomes were also prepared by encapsulating ³H Retinoic acid for the skin penetration studies.

Zein-Retinoic Acid Nanoemulsion Preparation.

Zein (13.5 mg) in ethanol (2 ml) and retinoic acid (1 mg) were dissolved in water (0.2 ml) and dispersed and sonicated for 20 seconds. The resulting mixture was added drop wise to a solution containing 15 ml of citrate buffer pH (7.4), squalene (0.5%=0.075 ml), and lecithin (65 mg) and sonicated for 5 min, with 10 sec on and 1 sec off cycle, 37% amplitude at 10° C. (ice bath). The alcohol was evaporated under magnetic stirring at room temperature (2-3 hrs). The emulsion was subjected to centrifugal filtration (MW 10000 Da), at 4000 rpm for 60 minutes; 4° C. the emulsion was stored at room temperature.

Size, Polydispersity Index and Zeta Potential Determination.

Nanoparticles (2 mg) and micelles (2 mg) were dispersed in 2 ml of purified water and bath sonicated for 1 min. They were then centrifuged at 10000 rpm for 5 min and diluted 50 times with distilled water. About 50 μl of the nanoemulsion was taken and dilated to 1 mL using distilled water. A liposome dispersion was diluted 100 times using purified water and probe sonicated for 1 min using 20% amplitude. The sizes, polydispersity indexes, and zeta potentials, were measured with a Malvern Zetasizer. The results are shown in Table 6.

TABLE 6
Characteristics of retinoic acid formulations.

| Retinoic | | | Zeta | |
|---|---|---|---|---|
| Acid | Size (nm) | PDI | Potential | % EE |
| Nano- | 303.7 ± 6.5 | 0.413 ± 0.021 | 2.71 ± 0.18 | 72.23 ± 0.65 |
| particles | | | | |
| Micelles | 84.85 ± 3.1 | 0.175 ± 0.032 | 18.6 ± 0.56 | 75.05 ± 5.67 |
| Liposomes | 305.4 ± 11.6 | 0.312 ± 0.024 | 5.61 ± 0.84 | 62.18 ± 3.61 |
| Nano- | 152.0 ± 3.6 | 0.175 ± 0.009 | 75.5 ± 0.72 | 81.4 ± 2.45 |
| emulsions | | | | |

Encapsulation Efficiency.

An aliquot of nanoparticles, micelles, nanoemulsion and liposomes were dissolved in 90% ethanol and further diluted with 50% ethanol and retinoic acid content was measured using HPLC. The chromatographic conditions were as follows: Column: C18 (150 mm length, 5μ); Mobile Phase: Acetonitrile (1.13 ml/min)-1% w/v Ammonium Acetate Buffer (0.12 ml/min); Detection wavelength: 340 nm; Injection volume: 50 μl: Run time: 10 min. Results may be seen in Table 7.

In vitro release in Phosphate Buffer (pH 7.4).

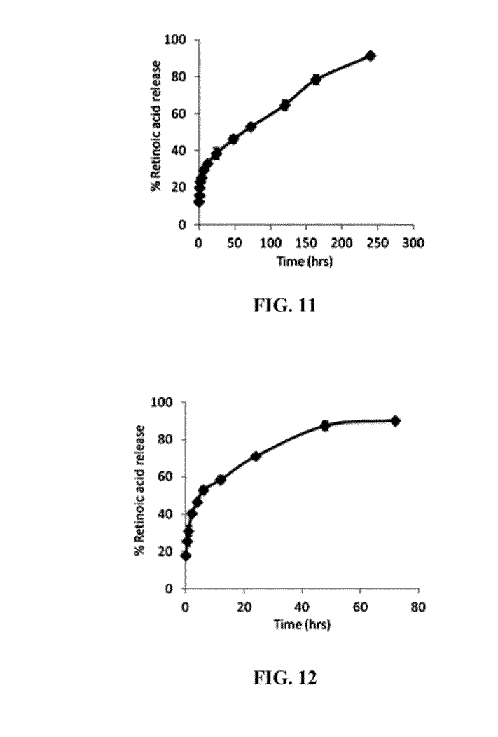

Release studies of the retinoic acid nanoparticles and micelles were carried out in phosphate buffer (pH 7.4) using vial method by placing them at 37° C. and 100 rpm. In each vial retinoic acid equivalent to 6 μg was taken. At each time interval the vial was centrifuged at 14000 rpm for 10 min and supernatant was analyzed for the retinoic acid content using a UV spectrophotometer at 350 nm. Simultaneously the amount of the retinoic acid in the pellet was estimated by dissolving in 1 ml of 90% ethanol. Similarly, release studies were also carried out the hot compound of the retinoic acid. Release studies were carried out in triplicate. Results are shown in FIGS. 11-12.

Atomic Force Microscopy Studies for Nanoemulsions.

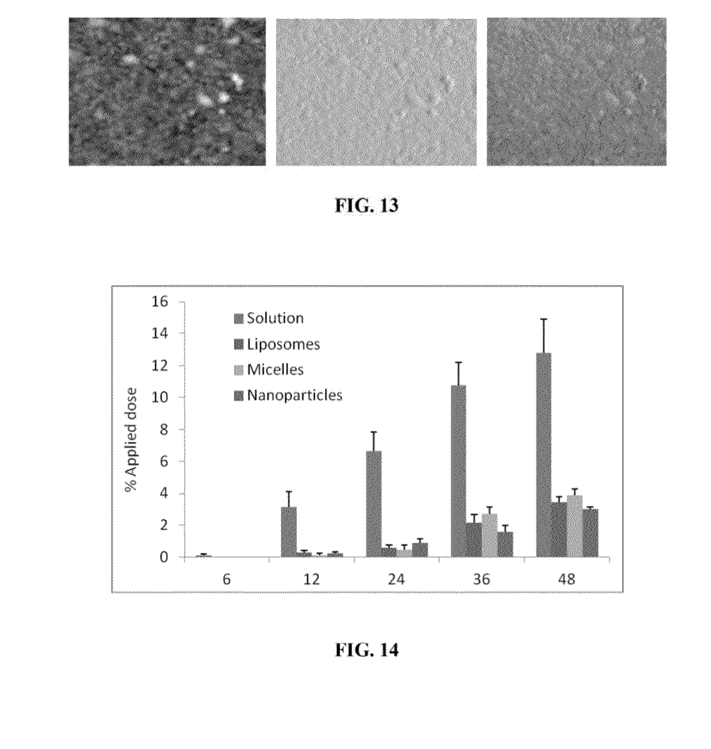

Approximately 20 μl of nanoemulsion was dispersed in 1 mL of distilled water, with or without zein. About 100 μl of supernatant was placed on a polyethylene amine coated glass cover slip and air dried. AFM images were collected in the scan area of 1 μm. Atomic force micrographs as shown in FIG. 13.

Skin Penetration Analysis.

Skin penetration analysis was carried out using porcine dermatomed skin (n=4) for 48 hrs at 37° C. under stirring using vertical Franz diffusion cell. Retinoic acid solution spiked with ³H retinoic acid (20% ethanol) was used, ³H retinoic acid encapsulated nanoparticles, micelles and liposomes were dispersed in 200 μl of phosphate buffer (pH 7.4) and taken in the donor compartment. The receptor compartment is filled with 5 ml of phosphate buffer (pH 7.4) for nanoparticles, micelles and liposomes. For retinoic acid solution 20% ethanol with phosphate buffer (pH 7.4) was used. At regular intervals 200 μl of sample from the receptor compartment was collected and replaced with the fresh medium. The amount of retinoic acid permeated across the skin and in the skin at the end of 48 hrs was determined using radiochemical method of analysis. Results are shown in FIG. 14.

Retinoic Acid Cream Formulations.

Plain cream preparation. Carbopol 981 NF (0.75% w/w) cream was prepared by soaking 75 mg of carbopol in 7 ml of purified water for 30 minutes. Later cream was triturated using a glass rod for 15-30 minutes until no aggregates of carbopol were seen.

HPMC K4M (1.5% w/w) cream was prepared by soaking 150 mg of HPMC in 7 ml of purified water for 30 minutes. Later cream was triturated using a glass rod for 15-30 minutes until no aggregates of HPMC were seen.

Retinoic Acid: Nanoparticles and Micelles Incorporated Cream Formulation.

700 mg of the carbopol and HPMC cream were taken and added with a weighed quantity of RA nanoparticles and micelles dispersed in 200 μl of phosphate buffer (pH 7.4). Further, pH was adjusted to 6.5 using triethanolamine and 0.1M HCl for carbopol and HPMC creams respectively.

Extraction of Retinoic Acid from HPMC and Carbopol Creams.

Known amounts of nanoparticle and micelle incorporated cream was weighed and dispersed in 90% ethanol using a bath sonicator for a period of 10 minutes. Later the dispersion was centrifuged at 10000 rpm for 10 minutes. The supernatant was filtered using 0.2 μm filter membrane and estimated the retinoic acid content using HPLC. Recovery of retinoic acid was found to be in the range of 90-94%.

Skin Penetration Studies of the Cream Formulations.

Skin penetration studies were carried out using porcine dermatome skin for a period of 48 hrs. Free retinoic acid (100 μg) wad disperse in 200 μl of 20% v/v ethanol in phosphate buffer (pH 7.4) was taken in the donor compartment; similarly in the receptor compartment also 20% v/v ethanol in phosphate buffer (pH 7.4) was taken. Whereas for nanoparticles and micelle dispersion (approximately 200 μl containing 100 μg of the retinoic acid in phosphate buffer (pH 7.4)), in case of nanoparticles and micelles incorporated in HPMC and carbopol cream (approximately 200 mg of the cream formulations containing 100 μg of the retinoic acid was taken in the donor compartment). In dispersion and cream formulations phosphate buffer (pH 7.4) was taken in the receptor compartment.

Sebum Diffusion Studies.

Artificial sebum was prepared using the following composition: squalene (15% w/w), paraffin wax (10% w/w), spermaceti wax (15% w/w), olive oil (10% w/w), cotton seed oil (25%) w/w), coconut oil (10% w/w), oleic acid (1.4% w/w), palmitic acid (5% w/w), palmitoleic acid (5% w/w), cholesterol (1.2% w/w) and cholesterol oleate (2.4% w/w). The ingredients were accurately weighed (%, w/w) in a glass beaker and heated at 60° C. for about 5-10 minutes with intermittent stirring until all the solids became a clear liquid. Retinoic acid transport through the artificial sebum is carried out in 24-well format (TRANSWELL®, Corning Incorporated, NY). The supporting membrane (polycarbonate membrane, pore size of 0.4 μm) of each insert was loaded with 2.5±0.2 mg of the artificial sebum. A 150 ρL aliquot of aqueous dispersion/solution of retinoic acid (equivalent to 50 μg of retinoic acid in buffer, retinoic acid in 50% ethanol in buffer, retinoic acid nanoparticles and retinoic acid micelles in citrate-phosphate buffer (CPB, pH 5.5)) was applied onto the insert and 1 mL of preheated (37° C.) 50% ethanol in the CPB was used as receiver solution. The study was carried out in an incubator at 125 rpm under 37° C. The complete receptor medium was replaced with fresh medium at predetermined intervals, i.e., 10, 20, 30, 40, 50, 60, 75, 90, 120 and 180 minutes after treatment. The retinoic acid content from the receptor solutions was analyzed using HPLC. The cumulative quantity of drug in the receiver compartment was plotted as a function of time. The flux value for a given experiment was obtained from the linear slope (steady-state portion) of the cumulative amount of drug permeated versus time curve. Results shown in Table 7.

TABLE 7
Sebum flux values from citrate-phosphate buffer (pH 5.5).

| Formulation | Flux (μg/cm²/min) |
|---|---|
| Retinoic acid | 0.154 ± 0.003 |
| Retinoic acid solution in 50% ethanol | 0.304 ± 0.023 |
| Retinoic acid Nanoparticle | 0.286 ± 0.021 |
| Retinoic acid Micelles | 0.273 ± 0.011 |

Retinoic Acid Stability Analysis.

Retinoic nanoparticles and micelles were prepared with and without BHT. Formulated nanoparticles, micelles, retinoic acid and its solution were charged for stability at room temperature and 40° C./75% RH.

For solid state stability retinoic acid nanoparticles, micelles were taken in glass vials. For liquid state stability retinoic acid nanoparticles and micelles were dispersed in water and taken in glass vials. Retinoic acid material as such was also charged for the stability; similarly retinoic acid was also dispersed in ethanol and charged for the stability. All the vials were sealed with parafilm and covered with aluminum foil. Each time point weighed/measured amount of retinoic acid formulations were taken and measured the drug content using HPLC. Results are shown in Tables 8-11.

HPLC Chromatographic conditions: Column: C18 (150 mm length, 5μ); Mobile Phase: Acetonitrile (1.13 ml/min)—1% w/v Ammonium Acetate Buffer (0.12 ml/min); Detection Wavelength: 340 nm; Run time: 10 min.

TABLE 8
Retinoic acid solid state stability at room temperature.

| | % Retinoic acid remaining | | | | |
|---|---|---|---|---|---|
| | | | Retinoic acid | | Retinoic acid |
| | | Retinoic acid | Nanoparticles | Retinoic acid | Micelles |
| Days | Retinoic acid | Nanoparticles | with BHT | Micelles | with BHT |
| 0 | 100 | 100 | 100 | 100 | 100 |
| 15 | 99.98 ± 1.61 | 99.72 ± 0.60 | 100.12 ± 1.85 | 100.13 ± 0.20 | 100.94 ± 0.08 |
| 30 | 82.94 ± 1.17 | 99.51 ± .40 | 100.24 ± 0.51 | 99.64 ± 0.82 | 98.89 ± 1.14 |
| 60 | 77.37 ± 0.60 | 97.89 ± 0.96 | 98.38 ± 0.69 | 98.36 ± 0.72 | 98.81 ± 0.32 |
| 90 | 71.75 ± 1.09 | 97.30 ± 1.26 | 97.17 ± 1.10 | 91.40 ± 1.17 | 93.71 ± 0.72 |

TABLE 9
Retinoic acid solid state stability at 40° C./75% RH.

| | % Retinoic acid remaining | | | | |
|---|---|---|---|---|---|
| | | | Retinoic acid | | Retinoic acid |
| | | Retinoic acid | Nanoparticles | Retinoic acid | Micelles |
| Days | Retinoic acid | Nanoparticles | with BHT | Micelles | with BHT |
| 0 | 100 | 100 | 100 | 100 | 100 |
| 15 | 89.27 ± 0.05 | 98.68 ± 1.01 | 99.20 ± 0.66 | 99.50 ± 1.62 | 98.98 ± 0.92 |
| 30 | 73.05 ± 2.63 | 98.68 ± 0.83 | 98.29 ± 1.09 | 97.84 ± 0.57 | 98.18 ± 0.58 |
| 60 | 67.33 ± 0.97 | 89.70 ± 2.60 | 94.51 ± 0.81 | 97.63 ± 1.32 | 97.42 ± 0.78 |
| 90 | 60.40 ± 1.14 | 89.24 ± 1.82 | 92.37 ± 2.41 | 88.59 ± 1.00 | 90.78 ± 0.64 |

TABLE 10
Retinoic acid liquid state stability at room temperature.

| | % Retinoic acid remaining | | | | |
|---|---|---|---|---|---|
| | | | Retinoic acid | | Retinoic acid |
| | | Retinoic acid | Nanoparticles | Retinoic acid | Micelles |
| Days | Retinoic acid | Nanoparticles | with BHT | Micelles | with BHT |
| 0 | 100 | 100 | 100 | 100 | 100 |
| 15 | 93.17 ± 0.70 | 98.42 ± 0.76 | 99.15 ± 0.10 | 98.16 ± 2.83 | 99.23 ± 1.49 |
| 30 | 64.35 ± 0.89 | 86.38 ± 1.25 | 91.73 ± 0.98 | 90.47 ± 1.35 | 95.72 ± 1.26 |
| 60 | 51.18 ± 1.66 | 85.76 ± 0.66 | 91.32 ± 1.38 | 89.75 ± 1.07 | 93.58 ± 1.22 |
| 90 | 40.78 ± 1.32 | 80.11 ± 0.82 | 89.34 ± 1.15 | 87.28 ± 0.67 | 91.24 ± 0.30 |

TABLE 11
Retinoic acid liquid state stability at 40° C./75% RH.

| | % Retinoic acid remaining | | | | |
|---|---|---|---|---|---|
| | | | Retinoic acid | | Retinoic acid |
| | | Retinoic acid | Nanoparticles | Retinoic acid | Micelles |
| Days | Retinoic acid | Nanoparticles | with BHT | Micelles | with BHT |
| 0 | 100 | 100 | 100 | 100 | 100 |
| 15 | 82.22 ± 0.77 | 98.85 ± 0.91 | 98.25 ± 0.51 | 98.08 ± 1.28 | 99.52 ± 1.74 |
| 30 | 60.12 ± 0.72 | 82.59 ± 1.05 | 86.42 ± 0.02 | 62.79 ± 1.58 | 67.61 ± 0.40 |
| 60 | 38.63 ± 0.39 | 67.78 ± 0.32 | 66.24 ± 1.06 | 30.02 ± 0.39 | 35.63 ± 0.46 |
| 90 | 22.25 ± 1.81 | 56.14 ± 1.20 | 56.84 ± 0.83 | 15.04 ± 0.92 | 18.82 ± 0.08 |

Retinoic Acid Cream Formulation Stability Analysis.

Retinoic nanoparticles and micelles creams were prepared using carbopol and HPMC bases with and without BHT. Formulated creams were charged for stability at refrigerator (2-8° C.), room temperature and 40° C./75% RH. Formulations were taken in the glass vials sealed with parafilm and covered with aluminum foil. Each time point known amount of nanoparticle and micelle incorporated cream was weighed and dispersed in 90% ethanol using bath sonicator for a period of 10 minutes. Later the dispersion was centrifuged at 10000 rpm for 10 minutes. Supernatant was filtered using 0.2 μm filter membrane and estimated the retinoic acid content using HPLC.

Formulation codes:

CN: RA Nanoparticles in Carbopol cream

CNB: RA Nanoparticles with BHT in Carbopol cream

CM: RA Micelles in Carbopol cream

CMB: RA Micelles with BHT in Carbopol cream

HN: RA Nanoparticles in HPMC cream

HNB: RA Nanoparticles with BHT in HPMC cream

HM: RA Micelles in HPMC cream

HMB: RA Micelles with BHT in HPMC cream

TABLE 12
Retinoic acid cream formulation stability at refrigerator conditions (2-8° C.).

| | % Retinoic acid remaining | | | | | | | |
|---|---|---|---|---|---|---|---|---|
| Days | CN | CNB | CM | CMB | HN | HNB | HM | HMB |
| 0 | 100 | 100 | 100 | 100 | 100 | 100 | 100 | 100 |
| 15 | 95.62 ± 0.78 | 95.26 ± 1.23 | 97.92 ± 2.26 | 96.28 ± 0.89 | 100.56 ± 1.83 | 100.67 ± 2.82 | 74.53 ± 1.03 | 66.82 ± 0.82 |
| 30 | 96.40 ± 9.18 | 95.44 ± 2.90 | 96.67 ± 2.33 | 95.33 ± 17.74 | 95.26 ± 3.67 | 99.34 ± 2.14 | 60.90 ± 1.19 | 55.93 ± 4.75 |
| 60 | 94.62 ± 1.96 | 94.59 ± 0.54 | 96.67 ± 1.42 | 99.34 ± 2.75 | 98.66 ± 0.79 | 98.80 ± 0.46 | 59.17 ± 1.88 | 51.61 ± 1.68 |
| 90 | 94.37 ± 0.09 | 94.55 ± 0.77 | 96.18 ± 1.63 | 99.93 ± 1.97 | 97.59 ± 1.82 | 98.52 ± 2.28 | 56.85 ± 4.47 | 52.83 ± 1.65 |

TABLE 13
Retinoic acid cream formulation stability at room temperature.

| | % Retinoic acid remaining | | | | | | | |
|---|---|---|---|---|---|---|---|---|
| Days | CN | CNB | CM | CMB | HN | HNB | HM | HMB |
| 0 | 100 | 100 | 100 | 100 | 100 | 100 | 100 | 100 |
| 15 | 93.33 ± 4.86 | 92.66 ± 1.33 | 93.84 ± 1.27 | 99.19 ± 1.18 | 100.84 ± 1.06 | 99.67 ± 1.60 | 77.37 ± 2.68 | 67.37 ± 1.72 |
| 30 | 93.57 ± 2.09 | 92.64 ± 0.57 | 91.14 ± 2.48 | 95.16 ± 1.81 | 90.53 ± 1.00 | 99.62 ± 3.40 | 73.44 ± 1.73 | 70.89 ± 5.09 |
| 60 | 91.05 ± 1.45 | 94.64 ± 1.39 | 91.92 ± 1.92 | 97.23 ± 3.09 | 95.25 ± 1.69 | 99.84 ± 1.74 | 71.56 ± 3.24 | 68.56 ± 0.19 |
| 90 | 91.38 ± 0.89 | 94.78 ± 7.08 | 91.64 ± 0.49 | 97.19 ± 0.84 | 93.75 ± 0.19 | 99.43 ± 3.40 | 72.46 ± 1.03 | 62.47 ± 1.05 |

TABLE 14
Retinoic acid cream formulation stability at 40° C./75% RH.

| | % Retinoic acid remaining | | | | | | | |
|---|---|---|---|---|---|---|---|---|
| Days | CN | CNB | CM | CMB | HN | HNB | HM | HMB |
| 0 | 100 | 100 | 100 | 100 | 100 | 100 | 100 | 100 |
| 15 | 86.28 ± 1.82 | 86.95 ± 1.25 | 66.40 ± 0.89 | 64.13 ± 1.06 | 82.78 ± 2.55 | 89.16 ± 1.11 | 25.43 ± 1.92 | 28.22 ± 2.08 |
| 30 | 64.64 ± 0.03 | 73.29 ± 0.95 | 48.96 ± 3.88 | 59.81 ± 0.42 | 60.36 ± 1.35 | 61.40 ± 2.95 | 9.14 ± 0.53 | 7.62 ± 0.48 |
| 60 | 48.54 ± 1.17 | 60.53 ± 0.89 | 46.73 ± 0.10 | 48.91 ± 1.92 | 54.05 ± 0.13 | 64.99 ± 0.78 | 4.77 ± 0.43 | 6.61 ± 0.71 |
| 90 | 45.83 ± 0.89 | 58.32 ± 4.81 | 37.72 ± 3.32 | 40.96 ± 3.60 | 47.19 ± 0.60 | 58.87 ± 1.57 | 4.093 ± 0.30 | 4.02 ± 2.49 |

Retinoic acid release studies from the in-house and marketed formulations.

Retinoic acid nanoparticles and micelles were incorporated in HPMC K4M (0.75% w/w) and prepared 0.1% w/w of retinoic nanoparticle and micelle cream. Marketed Formulation: RETIN-A MICRO™ Gel (0.1 w/w Retinoic acid) and RETIN-A CREAM™ (0.1 w/w Retinoic acid) were used for the analysis.

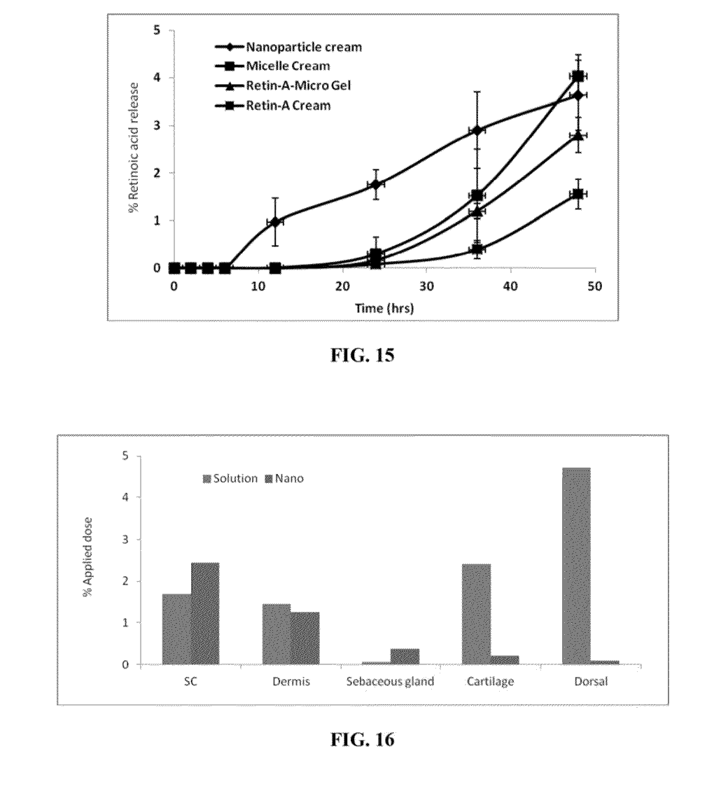

In vitro release analysis was carried out using vertical Franz diffusion cell. Donor and receptor compartments were separated using cellulose ester dialysis membrane (MWCO: 8000-1000; Spectrum Labs). In the receptor compartment 20% ethanol with PBS (pH 7.4) was used and in the donor compartment 100 mg of the formulation was used. Samples from the receptor compartment were withdrawn at predetermined intervals, i.e. 0, 2, 4, 6, 12, 24, 36 and 48 hrs and replaced with the fresh medium. Retinoic acid content from the samples was analyzed using HPLC. Results are shown in FIG. 15.

In vitro studies using hamster ear.

The excised hamster ear was placed on the Franz diffusion cell by exposing ventral surface towards the donor compartment. About 200 μl of the retinoic add (100 μg) in 20% ethanol with PBS (pH 7.4) and retinoic acid nanoparticles (retinoic acid equivalent to 100 μg) in PBS (pH 7.4) were taken in the donor compartment and treated for 24 hrs. At the end of the treatment period the amount of retinoic acid in the epidermis (ventral), cartilage, dorsal (whole skin), pilosebaceous (ventral) and dermis (ventral) were quantified using radiochemical analysis. Results can be seen in FIG. 16 and Table 15.

TABLE 15
Distribution of retinoic acid in various areas
of hamster ear at the end of 24 hrs treatment.

% Applied dose

| Compartment | Solution | Nanoparticles |
|---|---|---|
| SC | 1.692083 | 2.438539 |
| Dermis | 1.444046 | 1.250041 |
| Sebaceous gland | 0.066186 | 0.389305 |
| Cartilage | 2.408622 | 0.210989 |
| Dorsal | 4.71337 | 0.093241 |

Hamster Flank Skin Penetration (In Vitro) Analysis.

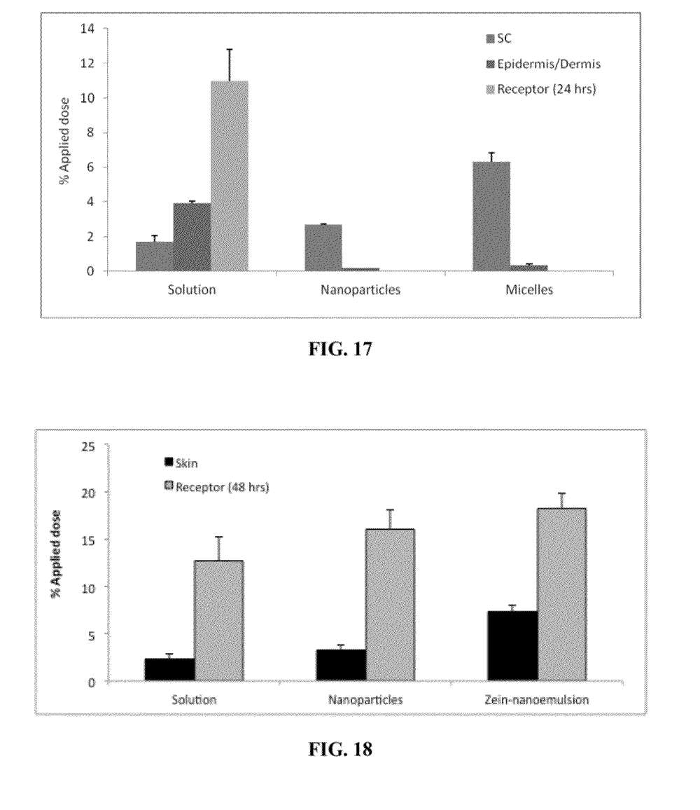

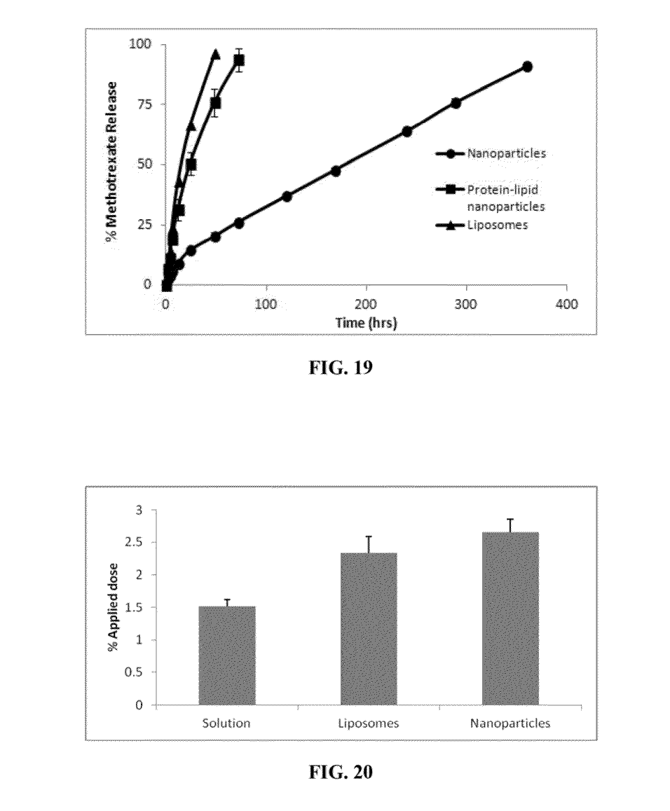

Flank skin was isolated and mounted on the Frantz diffusion cell by exposing epidermis towards the donor compartment. About 200 μl of the retinoic acid (100 μg) in 20% ethanol with PBS (pH 7.4) and Retinoic acid nanoparticles and micelles (retinoic acid equivalent to 100 μg) in PBS (pH 7.4) were taken in the donor compartment and treated for 24 hrs. At the end of the treatment period the amount of retinoic acid in the stratum corneum, epidermis/dermis and receptor compartment were quantified using radiochemical analysis. Results can be seen in FIG. 17.

In Vivo Analysis.

In vivo analysis of the marketed and in-house formulations was carried out using male Syrian hamsters. In this analysis 50 mg of the formulations (retinoic acid equivalent to 50 μg) were applied on the ventral surface with slight massage over 1 cm² area for 8 hrs. After 8 hrs hamsters were sacrificed and ears were processed to get the amount of drug present in the stratum corneum, ventral dermis, pilosebaceous glands, cartilage and dorsal dermis. Retinoic acid was extracted using 50% ethanol and estimated using HPLC. Results can be seen in Table 16.

TABLE 16
Retinoic acid (μg) distribution in various parts of hamster ear.

| | Retin-A | Retin-A | Micelle | Nanoparticle | Nanoemulsion |
|---|---|---|---|---|---|
| | Cream | Micro Gel | Cream | Cream | Cream |
| Stratum Corneum | 2.1 (0.47) | 2.02 (0.17) | 2.89 (0.40) | 2.59 (0.11) | 2.44 (0.40) |
| Ventral Dermis | 1.98 (0.26) | 1.74 (0.21) | 2.22 (0.21) | 1.33 (0.72) | 1.89 (0.12) |
| Sebum | 0.3 (0.09) | 0.49 (0.11) | 0.46 (0.05) | 0.26 (0.08) | 0.52 (0.05) |
| Cartilage | 0.50 (0.15) | 0.57 (0.07) | 0.36 (0.12) | 0.18 (0.07) | 0.21 (0.04) |
| Dorsal Dermis | 0.26 (0.10) | 0.31 (0.11) | 0.30 (0.13) | 0.11 (0.03) | 0.29 (0.05) |
| Pilosebaceous dermis | 0.15 (0.06) | 0.29 (0.09) | 0.21 (0.01) | 0.29 (0.27) | 0.27 (0.02) |
| ratio | | | | | |
| Total retinoic acid | 5.16 (0.41) | 5.15 (0.27) | 6.26 (0.34) | 4.49 (0.83) | 5.37 (0.48) |
| retained in the skin | | | | | |

Values are mean ± SD (n = 4).

### Example 4

### Zein-Methotrexate (MTX) Analysis

Zein-MTX Nanoparticle and Liposome Preparations.

Zein-MTX nanoparticles were made in a similar fashion as those made in Example 2 above, except that 1 mg of MTX was used in place of retinoic acid. In the same way, MTX containing liposomes were prepared as recited above for retinoic acid containing liposomes, except that 1 mg of MTX in 5 ml of phosphate buffer (pH 7.4) was added to the dry film prior to rotavapor treatment and separation steps.

Zein-MTX Nanoparticles vs. Liposome MTX.

Nanoparticles (2 mg) were dispersed in 1 ml of purified water and probe sonicated for 1 min using 20% amplitude. Subsequently, the sonicated particles were diluted 10 times with distilled water and properties were measured using a Malvern Zetasizer.

Liposome dispersions were diluted 20 times using purified water and probe sonicated for 1 min using 20% amplitude. Subsequently, the sonicated particles were measured using a Malvern Zetasizer. The results of the measurements may be seen in Table 17.

TABLE 17
Characteristics of MTX nanoparticles and liposomes.

| | | | Zeta | |
|---|---|---|---|---|
| Formula- | Particle size | | Potential | |
| tion | (nm) | PDI | (mv) | % EE |
| Nano- | 239.5 ± 13.1 | 0.27 ± 0.02 | −18.56 ± 0.07 | 43.90 ± 1.23 |
| particles | | | | |
| Liposomes | 266.3 ± 17.2 | 0.37 ± 0.02 | −15.29 ± 0.09 | 13.53 ± 1.26 |

PDI—polydispersity index; EE—encapsulation efficiency: Nanoparticles are composed of zein/lecithin/PLURONIC F68.

Entrapment Efficiency.

Nanoparticles (1 mg) were dispersed in 1 ml of 90% ethanol, sonicated and vortexed for 2 mm. The resulting solution was centrifuged at 5000 rpm for 5 min. The supernatant was collected and methotrexate content measured using a UV spectrophotometer at 300 nm.

Liposome dispersions (50 μl) were diluted to 1 ml with 50% ethanol, sonicated and vortexed for 2 min. The resulting solution was centrifuged 5000 rpm for 5 min. The methotrexate content for liposomes was measured as above. Results may be seen in Table 17.

Release Analysis.

Release analysis of the methrotrexate liopsomes and nanoparticles was carried out in phosphate buffer (pH 7.4) using dialysis method (n=3). Aliquots of methotrexate liposomes and nanoparticles were disperse in 2 ml of phosphate buffer (pH 7.4) and transferred to separate dialysis tubes. The dialysis tubes were placed in a reservoir compartment containing 15 ml of phosphate buffer (pH 7.4) and placed at 37° C. under 200 rpm. At each interval, 1 ml of sample was collected from the reservoir compartment and replaced with fresh 1 ml of phosphate buffer (pH 7.4). Methotrexate content was measured using a UV spectrophotometer at 300 nm. The results may be seen in FIG. 18.

Porcine In Vitro Skin Penetration Analysis (6 hrs Treatment).

Skin penetration analysis was carried out using full thickness porcine skin (n=3) for 6 hrs at 37° C. under stirring using vertical Franz diffusion cell. The encapsulated methotrexate solution was spiked with ³H methotrexate, where the ³H spiked liposomes and nanoparticles were dispersed in 400 μl of phosphate buffer (pH 7.4) and taken in the donor compartment. The receptor compartment was filled with 5 ml of phosphate buffer (pH 7.4).

At the end of 6 hrs, 200 μl of sample from the receptor compartment was collected and analyzed for the methotrexate content. Simultaneously, the amount of methotrexate in the skin was determined using radiochemical method of analysis. Results are shown in FIG. 20.

Porcine and Human Skin Penetration Studies (48 hrs Treatment).

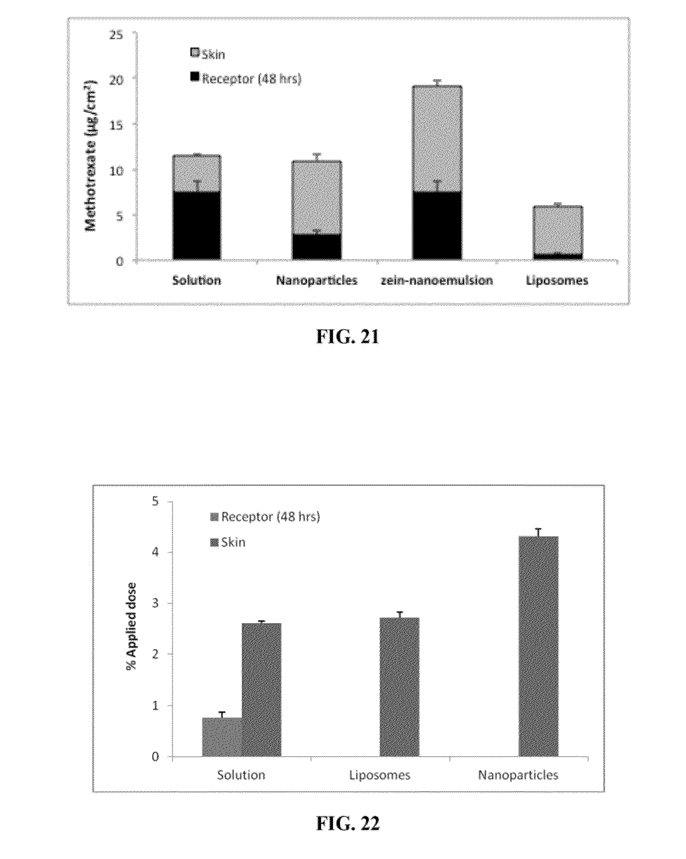

The method was similar to that for the 6 hour treatment except that at the end of 48 hours the amount of methotrexate was determined. Results may be seen in FIG. 21.

A similar analysis was carried out using human skin. Results may be seen in FIG. 22.

Skin Disposition Analysis.

Skin disposition studies were carried out using full thickness human skin (n=4) for 48 hrs at 37° C. under stirring using vertical Frantz diffusion cell.

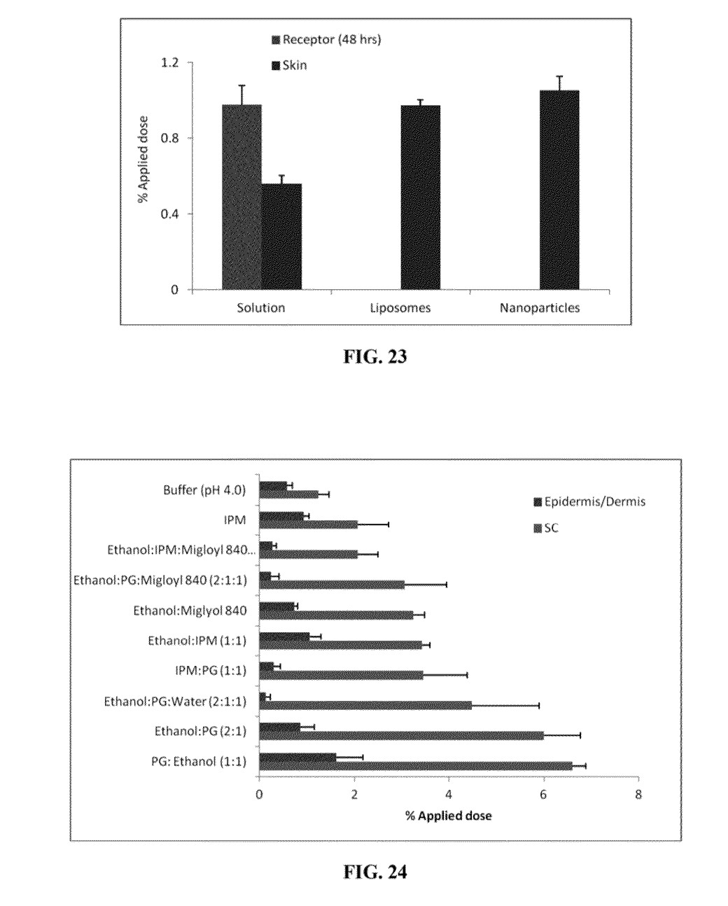

The encapsulated methotrexate solution was spiked with ³H methotrexate, where the ³H spiked liposomes and nanoparticles were dispersed in 400 μl of phosphate buffer (pH 7.4) and taken in the donor compartment treated with the skin for 6 hrs. After 6 hrs formulations were withdrawn and analysis was continued for 48 hrs. The receptor compartment was filled with 5 ml of phosphate buffer (pH 7.4), and at regular intervals, 200 μl of sample from the receptor compartment was collected and replaced with the fresh phosphate buffer (pH 7.4). The amount of methotrexate permeated across the skin and in the skin at the end of 48 hrs was determined using radiochemical method of analysis. Results may be seen in FIG. 23.

MTX nanoemulsions were prepared as follows: MTX (10 mg) and zein (15 mg) were dissolved in 2.2 ml of 90% ethanol. The hydroalcoholic solution was added to citrate buffer (pH 7.4) containing squalene (0.075 ml), lecithin (65 mg) under probe sonication and stirred in a magnetic stirrer for 3 hrs to remove alcohol. Free MTX was separated using centrifugal filter (MWCO: 10 kDa). The conventional zein-lecithin-PLURONIC F68 was prepared using a similar procedure as described above. The liposomes were prepared using cholesterol and lecithin in an organic solvent and the solvent was evaporated in a rotary evaporator followed by hydration of the film using an aqueous MTX solution.

As shown in Table 18, the nanoemulsions showed the lowest particle size compared to liposomes and zein-lecithin-PLURONIC nanoparticles.

TABLE 18
Characteristics of MTX nanocarriers and liposomes.

| | | | Zeta | |
|---|---|---|---|---|
| Formula- | Particle size | | Potential | |
| tion | (nm) | PDI | (mv) | % EE |
| Nano- | 239.5 ± 13.1 | 0.27 ± 0.02 | −18.56 ± 0.07 | 43.90 ± 1.23 |
| particles | | | | |
| Nano- | 214.3 ± 8.4 | 0.12 ± 0.09 | −65.23 ± 1.90 | 87.6 ± 2.10 |
| emulsions | | | | |
| Liposomes | 266.3 ± 17.2 | 0.37 ± 0.02 | −15.29 ± 0.09 | 13.53 ± 1.26 |

PDI—polydispersity index; EE—encapsulation efficiency: Nanoparticles are composed of zein/lecithin/PLURONIC F68; nanoemulsions comprise of zein/lecithin/squalene.

Further the size was much more uniform in case of nanoemulsions as evidenced from the low polydispersity index (PDI). The zeta potential was high in case of nanoemulsions signifying the high physical stability of these nanocarriers. Encapsulation efficiency was determined by UV analysis at 300 nm after digestion of the particles using hydroalcoholic solution. As can be seen from the Table 18, the zein nanoemulsions showed 6-fold higher encapsulation efficiency (EE) than liposomes and 2 fold higher MTX encapsulation than nanoparticles (Table 19). In vitro release of MTX from the formulations in phosphate buffer (pH 7.4) was studied using UV-spectrophotometry.

As can be seen from FIG. 23, the zein-lecithin-PLURONIC nanoparticles sustained MTX release longer than liposomes, while the MTX release from nanoemulsions was sustained longer than liposomes but shorter than zein-lecithin PLURONIC nanoparticles.

The in-vitro skin penetration of MTX formulations were analyzed using vertical Franz diffusion cell using excised porcine skin. The nanoemulsions showed 3 fold higher skin penetration/retention than free MTX solution and the skin penetration/retention was 2 fold higher than liposomes.

Compared to the zein-lecithin-PLURONIC nanoparticles, the nanoemulsions showed 1.4 times higher skin penetration of MTX, while the MTX in the receptor was comparable to free MTX solution.

By altering the zein/lecithin/squalene ratio, the skin retention can be further increased, while minimizing the drug penetration across the skin into systemic circulation.

### Skin Penetration of Methotrexate Using Various Solvents.

Skin penetration analysis was carried out using full thickness porcine skin (n=3) for 6 hrs at 37° C. under stirring using vertical Franz diffusion cell. Methotrexate (10 mg/ml) solution was prepared using various solvents; i.e., phosphate buffer (pH 4.0), propylene glycol:ethanol (1:1), ethanol:IPM (1:1), propylene glycol:phosphate buffer (1:1) (pH 3.85), ethanol:phosphate buffer (1:1) (pH: 4.72), ethanol:migloyl 840 (1:1), IPM (100%), IPM:PG (1:1), ethanol:PG (2:1), ethanol:PG:water (2:1:1), ethanol:PG:migloyl 840 (2:1:1), ethanol:IPM:migloyl 840 (2:1:1).

The methotrexate solutions were spiked with ³H methotrexate, where about 200 μl of the above solutions were taken in the donor compartment and treated for 6 hrs with the skin. At the end of 6 hrs, the amount of methotrexate in the stratum corneum (removed using scotch tape), epidermis/dermis, and receptor compartment was analyzed using radioactive scintillation counter. Results may be seen in FIG. 24.

Comparison Between Porcine and Human Skin.

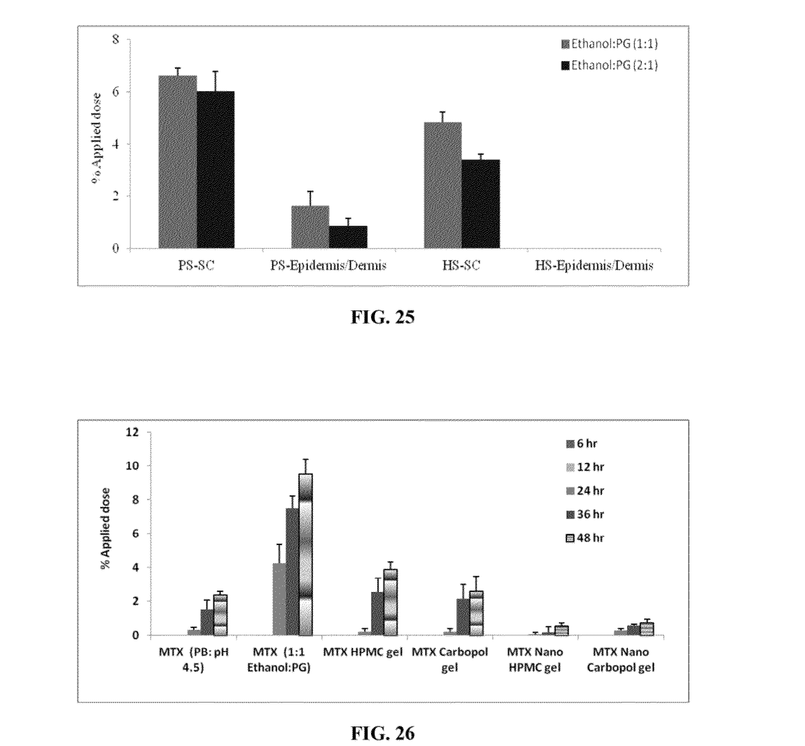

Methotrexate (10 mg/ml) solution was prepared using ethanol:PG (1:1 and 2:1). About 200 μl of the above solutions were taken in the donor compartment and treated for 6 hrs with the porcine (PS) and human (HS) full-thickness skin. At the end of 6 hrs, the amount of methotrexate in the stratum corneum (removed using scotch tape), epidermis/dermis and receptor compartment was analyzed using a scintillation counter. The results may be seen in FIG. 25.

### Example 5

### Methotrexate Transdermal Delivery Optimization

The skin penetration analysis was performed using a vertical Franz diffusion cell apparatus. The receptor compartment was filled with approximately 5 mL of receptor medium (Phosphate Buffer Saline (pH 7.4)). The receptor medium was maintained at 37° C. for the duration of the study using a circulating water bath equipped with a heater. The receptor medium was continually stirred using a magnetic stir bar. The full thickness porcine skin was thawed to room temperature and mounted on the Franz diffusion cells with the stratum corneum facing the donor compartment. 200 μl of Methotrexate (Cold compound 10 mg/mL and hot compound 1 μl of ³H MTX) were prepared in various solvents as shown in the Table 19 were taken in the donor compartment and covered with parafilm throughout the study. An aliquot (200 μl) was removed from the receptor medium at the following time points 0, 2, 4, 6, 12, 24, 36, 48 hours (n=4). The volume displaced was replaced with 200 μl of fresh receptor medium. Collected samples were mixed with 2 ml of scintillation cocktail and radioactive counts were measured using liquid scintillation counter.

TABLE 19
Solvent composition for the penetration studies.

| Exp. | | |
|---|---|---|
| No | Solvent Combination | Vehicle ratio |
| 1 | Ethanol:PG | 1:1 |
| 2 | Ethanol:PG:Santalol | 5:4:1 |
| 3 | Ethanol:PG:Eucalyptol | 5:2.5:2.5 |
| 4 | Ethanol:PG:Ethyl acetate | 6:2:2 |

TABLE 20
Shows the values of TEWL

| Exp. | | |
|---|---|---|
| No | Solvent Combination | TEWL Values |
| 1 | Ethanol:PG-1 | 8.5 |
| | Ethanol:PG-2 | 8.4 |
| | Ethanol:PG-3 | 7.6 |
| | Ethanol:PG-4 | 8.7 |
| 2 | Ethanol:PG:Santalol-1 | 6.9 |
| | Ethanol:PG:Santalol-2 | 7.6 |
| | Ethanol:PG:Santalol-3 | 8.2 |
| | Ethanol:PG:Santalol-4 | 6.7 |
| 3 | Ethanol:PG:Eucalyptol-1 | 7.3 |
| | Ethanol:PG:Eucalyptol-2 | 7.6 |
| | Ethanol:PG:Eucalyptol-3 | 8.4 |
| | Ethanol:PG:Eucalyptol-4 | 6.9 |
| 4 | Ethanol:PG:Ethyl acetate-1 | 6.7 |
| | Ethanol:PG:Ethyl acetate-2 | 7.4 |
| | Ethanol:PG:Ethyl acetate-3 | 7.1 |
| | Ethanol:PG:Ethyl acetate-4 | 7.8 |

TABLE 21
Shows the Flux, Lag Time, and Cumulative Amount for Methotrexate from various solvent combinations.

| | Ethanol:PG | | Ethanol:PG:Santalol | | Ethanol:PG:Eucalyptol | | Ethanol:PG:Ethyl acetate | |
|---|---|---|---|---|---|---|---|---|
| Time (hrs) | (n = 4) | | (n = 4) | | (n = 4) | | (n = 4) | |
| Flux (μg/cm²/hr) | 2.93 | (0.25) | 6.34 | (2.54) | 13.05 | (3.63) | 3.82 | (0.62) |
| Lag Time (hours) | 0.93 | (0.36) | 8.91 | (6.35) | 15.75 | (6.13) | 10.91 | (3.13) |
| Cumulative Amount - | 82.69 | (6.51) | 185.20 | (80.08) | 763.38 | (166.50) | 196.53 | (22.23) |
| 48 hrs (μg/cm²) | | | | | | | | |

TABLE 22
Shows the average cumulative amounts of the Methotrexate
Average Cumulative Amount (μg/cm²)
(Std. Dev.)

| Time | Ethanol:PG | | Ethanol:PG:Santalol | | Ethanol:PG:Eucalyptol | | Ethanol:PG:Ethyl acetate | |
|---|---|---|---|---|---|---|---|---|
| (hrs) | (n = 4) | | (n = 4) | | (n = 4) | | (n = 4) | |
| 0 | 0 | | 0 | | 0 | | 0 | |
| 2 | 0.56 | (0.65) | 1.21 | (1.23) | 11.79 | (7.84) | 3.56 | (1.13) |
| 4 | 3.98 | (1.06) | 8.55 | (5.60) | 57.68 | (32.36) | 20.28 | (3.31) |
| 6 | 8.00 | (0.46) | 17.11 | (9.63) | 118.64 | (53.24) | 31.34 | (5.30) |
| 12 | 21.69 | (1.46) | 66.86 | (37.83) | 354.63 | (63.93) | 82.94 | (13.30) |
| 24 | 47.76 | (3.02) | 131.69 | (51.80) | 510.74 | (80.01) | 140.62 | (24.91) |
| 36 | 66.75 | (4.25) | 164.02 | (69.63) | 671.52 | (141.99) | 174.70 | (25.21) |
| 48 | 82.69 | (6.51) | 185.20 | (80.08) | 763.38 | (166.50) | 196.53 | (22.23) |

Conclusions: Among the solvents screened Ethanol:Propylene Glycol:Eucalyptol (5:2.5:2.5) has shown better flux and penetration of methotrexate compared to all other combinations (see, e.g., Table 21).

### Example 6

### Methotrexate (MTX) Gel Formulations

Simple MTX Gel Formulation.

Carbopol 981 NF (0.75% w/w) gel was prepared by soaking 75 mg of carbopol in 7 ml of purified water for 30 minutes. Later, the gel was triturated using a glass rod for 15-30 minutes until there are no aggregates of carbopol. From the triturated material, 700 mg of the gel was taken and added with 1 mg of methotrexate in 200 μl of ethanol:PG (1:1 ratio). Further, the pH of the gel formulation was adjusted to pH 4.5 using triethanolamine.

HPMC K4M (1.5% w/w) gel was prepared by soaking 150 mg of HPMC in 7 ml of purified water for 30 minutes. Later, the gel was triturated using a glass rod for 15-30 minutes until there are no aggregates of HPMC. From the triturated material, 700 mg of the gel was taken and added with 1 mg of methotrexate in 200 μl of ethanol:PG (1:1 ratio). Further, the pH of the gel formulation was adjusted to pH 4.5 using 0.1 M HCl.

Methotrexate-Nanoparticle Incorporated Gel Formulation.

700 mg of the carbopol and KPMC gels were taken and added with 180 mg of MTX nanoparticles dispersed in 200 μl of phosphate buffer (pH 7.4). Further, the pH was adjusted using triethanolamine and 0.1M HCl for carbopol and HPMC gels respectively.

Skin Penetration Analysis of the Gel Formulations.

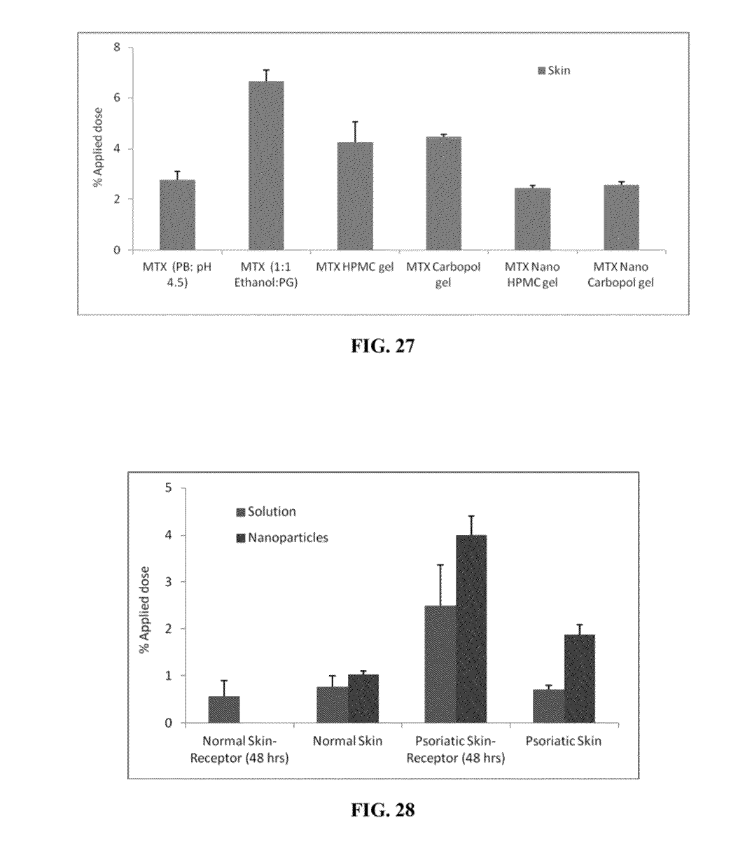

Skin penetration was carried out using porcine full-thickness skin for a period of 48 hrs. In the case of methotrexate in phosphate buffer (pH 7.4) and ethanol:PG (1:1), 200 μl (equivalent to 200 μg of MTX) of was taken in the donor compartment. Whereas for simple formulations, about 200 mg of the gel formulation (equivalent to 200 μg of MTX) were taken each donor compartment, and for the nanoparticle-incorporated formulations, about 300 mg of the gel formulation (equivalent to 200 μg of MTX) was taken in each donor compartment. The results may be seen in FIGS. 26 and 27.

Psoriatic and Non-Psoriatic Human Skin Analysis.

Skin penetration analysis was carried out in vitro for a period of 48 hrs using human full-thickness psoriatic and non-psoriatic skin from same volunteer skin. The encapsulated methotrexate solution was spiked with ³H methotrexate, where the ³H spiked nanoparticles were dispersed in 400 μl of phosphate buffer (pH7 7.4) and taken in the donor compartment. The receptor compartment was filled with 5 ml of phosphate buffer (pH 7.4). At regular intervals 200 μl of sample from the receptor compartment was collected and replaced with fresh phosphate buffer (pH 7.4). The amount of methotrexate that permeates across the skin and deposits in the skin at the end of 48 hrs was determined using radiochemical method of analysis. Results may be seen in FIG. 28.

Summary.

Nanoparticle formulations and topical formulations with penetration enhancers were developed to improve the skin penetration and achieve therapeutic concentrations in the skin to treat psoriasis. Zein based core-shell nanoparticles showed higher drug encapsulation, better sustained release and higher skin penetration than liposomes in psoriatic and non-psoriatic human skin. Further formulations with penetration enhancers also increased the skin penetration of MTX.

### Example 7

### Application of Zein Nanocarriers for Delivery of Benzoyl Peroxide.

Benzoyl Peroxide Nanocarrier Formulations.

Method of Preparation of Nanoparticles: zein (13.5 mg) and benzoyl peroxide (50 mg) were dissolved in Ethanol (90%). Lecithin (65 mg) and PLURONIC F68 (130 mg) were dispersed in 15 ml citrate buffer (pH 7.4) the ethanol solution was added drop wise to citrate buffer solution under probe sonication (10 min, 37% amplitude) with 10 sec on and 1 sec off cycle. Further, the solution was kept under stirring at room temperature for about 3 hrs. The entrapped and free benzoyl peroxide were separated using centrifugal filters, and the benzoyl peroxide nanoparticles dispersion were stored under refrigeration until use. Similarly benzoyl peroxide nanoparticles were prepared by incorporating 10 μl of hot ¹⁴C benzoyl peroxide along with 50 mg of benzoyl peroxide.

Method of Preparation of Nanoemulsions: zein (25 mg) and benzoyl Peroxide (50 mg) were dissolved en ethanol (90%). Lecithin (65 mg) and squalene (0.075 mg) were dispersed in 15 ml citrate buffer (pH 7.4). The ethanol solution was added drop wise to citrate buffer solution under probe sonication (10 mg. 37% amplitude) with 10 sec on and 1 sec off cycle. Further, the solution was kept under stirring at room temperature for about 3 hrs. The entrapped and free benzoyl peroxide were separated using centrifugal filters, and the benzoyl peroxide nanoemulsion was stored under refrigeration until use. Similarly benzoyl peroxide nanoemulsions were prepared by incorporating 10 μl of hot ¹⁴C benzoyl peroxide along with 50 mg of benzoyl peroxide. p Method of Preparation of Micelles: PEG-Zein (100 mg) and benzoyl peroxide (1 mg) were dispersed in ethanol (90%) and incubated for 10 hrs at 37° C. under stirring. Further, the dispersion was dialyzed against water for about 6-8 hrs by changing the water 6-8 times. The micelle dispersion was lyophilized and stored under refrigeration until use.

Size, polydispersity index and zeta potential determination: nanoparticles (50 μl), nanoemulsions (squalene) (50 μl) and nanomicelles (2 mg) were dispersed in 2 ml of purified water separately, vortex for 1 min. Further, the size, polydispersity index and zeta potential were measured with a Malvern Zetasizer.

Encapsulation Efficiency: known amount of nanoparticles, nanoemulsions and micelles, were dissolved in 90% ethanol and suitably diluted. Benzoyl peroxide content was measured using HPLC.

Optimized HPLC conditions: Mobile Phase (70:30); Injection Volume: 25μ; Wave length: 238 nm; Flow rate: 1 mL/min. Column: C18 (150 mm length, 5μ).

Skin Penetration Analysis: skin penetration analysis was carried out using porcine dermatome skin (n=4) for 48 hrs at 37° C. under stirring using vertical Franz diffusion cell. Benzoyl peroxide (2.5 mg/mL) solution spiked with ¹⁴C Benzoyl peroxide (40:60 v/v ethanol:PBS pH (7.4) were used), ¹⁴C benzoyl peroxide encapsulated nanoparticles (Benzoyl peroxide equivalent to 2.5 mg/mL) and nanoemulsions (Benzoyl peroxide equivalent to 2.5 mg/mL) were prepared and about 400 μl were taken in donor compartment. The receptor compartment was filled with 5 ml of 20:80 v/v ethanol:PBS pH (7.4). At regular intervals 200 μl of sample from the receptor compartment was collected and replaced with the fresh medium. The amounts of benzoyl peroxide permeating across the skin and depositing in the skin at the end of 48 hrs was determined using radiochemical method of analysis.

A wide variety of drug molecules with varying physico-chemical properties can be encapsulated in protein-lipid nanoparticles as shown in Table 23. As can be seen from the Table 23, a high encapsulation efficiency can be achieved for both hydrophilic and hydrophobic drug molecules. In addition the nanoparticles can improve the chemical stability of retinoic acid and benzoyl peroxide (anti-acne drugs). Further the encapsulation avoids the skin irritation issues of retinoic acid and benzoyl peroxide.

TABLE 23
Characteristics of molecules encapsulated in nanoemulsions.

| | Molecular | | Particle size | |
|---|---|---|---|---|
| Drugs | weight (Da) | Log P | (nm) | % EE |
| Methotrexate | 454 | −1.8 | 214.3 ± 8.4 | 87.60 ± 2.1 |
| Benzoyl peroxide | 242.23 | 3.48 | 208.5 ± 6.7 | 83.66 ± 3.81 |
| Retinoic acid | 300.44 | 6.83 | 152 ± 3.6 | 81.4 ± 2.45 |

PDI—polydispersity index; EE—encapsulation efficiency: nanoemulsions comprise zein/lecithin/squalene.

Zein-emulsions showed high drug encapsulation compared to zein-lecithin-PLURONIC nanoparticles and PEG-zein micelles. Further the size of the zein-nanoemulsions was smaller than the zein-lecithin-PLURONIC nanoparticles (Table 24). Zein-nanoemulsions resulted in 2 fold increase in skin penetration of BPO compared to zein-lecithin-PLURONIC nanoparticles and 3 fold higher skin penetration than BPO solution (FIG. 18).

TABLE 24
Characteristics of BPO nanocarriers.

| BPO | | | Zeta | |
|---|---|---|---|---|
| formula- | Particle size | | Potential | |
| tions | (nm) | PDI | (mv) | % EE |
| Nano- | 439.1 ± 20.6 | 0.467 ± 0.06 | −67.2 ± 2.3 | 66.90 ± 1.51 |
| particles | | | | |
| Micelles | 126.7 ± 4.6 | 0.242 ± 0.02 | 8.92 ± 3.1 | 6.63 ± 0.53 |
| Nano- | 208.5 ± 6.7 | 0.284 ± 0.02 | −55.4 ± 5.4 | 83.66 ± 3.81 |
| emulsions | | | | |

PDI—polydispersity index; EE—encapsulation efficiency: Nanoparticles are composed of zein/lecithin/PLURONIC F68; nanoemulsions contain zein/lecithin/squalene.

Summary.

BPO can be mixed with various zein nanocarriers, including that BPO zein-nanoemulsions showed higher drug encapsulation and higher skin penetration than zein nanoparticles and zein nanomicelles.

Although the invention has been described with reference to the above examples, it will be understood that modifications and variations are encompassed within the spirit and scope of the invention. Accordingly, the invention is limited only by the following claims.

We claim herein:

1. A composition comprising a zein shell-core nanoparticle, zein-nanoemulsion or zein-nanomicelle containing a medicament, wherein said medicament is selected from retinoic acid, benzoyl peroxide (BPO) or methotrexate (MTX).

2. The composition of claim 1, wherein the zein core-shell nanoparticle further comprises a phospholipid and a surfactant.

3. The composition of claim 2, wherein the phospholipid is lecithin and the surfactant is a block co-polymer.

4. The composition of claim 3, wherein the block co-polymer is a poloxamer.

5. The composition of claim 1, wherein the zein-micelle comprises a polyethylene glycol (PEG).

6. The composition of claim 1, wherein the zein-nanoemulsion further comprises a phospholipid and a triterpene.

7. The composition of claim 6, wherein the phospholipid is lecithin and the triterpene is squalene or squalene monohydroperoxide, and wherein the zein-nanoemulsion exhibits a zeta-potential greater than about ±50.

8. The composition of claim 7, wherein the ratio of zein to medicament is between about 1:3.7 to about 13.5:1.

9. The composition of claim 1, wherein said zein shell-core nanoparticle, zein-nanoemulsion or zein-nanomicelle containing said medicament is formulated as a gel or a cream, and optionally contains one or more compounds selected from the group consisting of salicylic acid, sulfur, erythromycin or clindamycin, adapalene.

10. A method of treating a dermatological condition in a subject in need thereof comprising administering a cream or gel composition containing a zein shell-core nanoparticle, zein-nanoemulsion or zein-nanomicelle and a medicament selected from retinoic acid, benzoyl peroxide or methotrexate (MTX).

11. The method of claim 10, wherein the dermatological condition is selected from the group consisting of acne, seborrhetic eczema, androgenic alopecia, alopecia areata, folliculitis, hyperplastic lesions, psoriasis vulgaris, guttate psoriasis, inverse psoriasis, pustular psoriasis, and erythrodermic psoriasis, and wherein the hyperplastic lesions are selected from the group consisting of basaloid follicular hamartoma, basaloid epidermal proliferation, overlying dermal mesenchymal lesions, trichofolliculoma, sebaceous trichofolliculoma, folliculosebaceous cystic hamartoma, trichodiscoma/fibrofolliculoma, pilar sheath acanthoma, sebaceous hyperplasia, nevus sebaceous of Jadassohn, trichofolliculoma, desmoplastic trichoepithelioma, trichoblastoma, trichoblastic fibroma, trichoadenoma, proliferating trichilemmal cyst/pilar tumor, tricholemmoma, desmoplastic trichlilemmoma, pilomatricoma/proliferative pilomatricoma, sebaceous adenoma, sebaceous/sebaceous epithelioma, trichilemmal carcinoma, trichoblastic carcinoma, malignant proliferating trichilemmal cyst, pilomatrix carcinoma, sebaceous gland carcinoma, basil cell carcinoma with sebaceous differentiation, scleroderma, and skin adnexal tumors.

12. The method of claim 10, wherein the zein core-shell nanoparticle further comprises a phospholipid and a surfactant, wherein said nanoparticle partitions said medicament into a pilosebaceous unit.

13. The method of claim 12, wherein the phospholipid is lecithin and the surfactant is a block co-polymer.

14. The method of claim 13, wherein the block co-polymer is a poloxamer.

15. The method of claim 10, wherein the zein-micelle comprises a polyethylene glycol (PEG), wherein said zein-micelle partitions said medicament into a pilosebaceous unit.

16. The method of claim 10, wherein the zein-nanoemulsion further comprises a phospholipid and a triterpene, wherein said nanoemulsion partitions said medicament into a pilosebaceous unit.

17. The method of claim 16, wherein the phospholipid is lecithin and the triterpene is squalene or squalene monohydroperoxide (Sq-OOH), and wherein the zein-nanoemulsion exhibits a zeta-potential greater than about ±50.

18. The method of claim 10, wherein said zein shell-core nanoparticle, zein-nanoemulsion or zein-nanomicelle containing said medicament is formulated as a gel or a cream, and optionally contains one or more compounds selected from the group consisting of salicyclic acid, sulfur, erythromycin or clindamycin, and adapalene.

19. A formulation comprising a zein-nanoemulsion containing a phospholipid, a squalene or squalene monohydroperoxide (Sq-OOH), and a medicament selected from retinoic acid, benzoyl peroxide or methotrexate (MTX), wherein said zein-nanoemulsion exhibits a zeta-potential greater than about ±50.

20. The formulation of claim 10, wherein said formulation is a cream.

21. The formulation of claim 19, wherein said formulation is a gel.

22. A method of transdermally delivering methotrexate (MTX) to treat a condition in a subject in need thereof comprising administrating a composition comprising MTX in combination with two or more solvents selected from the group consisting of ethanol, transcutol, IPM, migloyl, phosphate buffer (4.0), santalol, eucalyptol, propylene glycol (PG), ethyl acetate, and combinations thereof.

23. The method of claim 22, wherein the MTX is combined with ethanol, PG and eucalyptol.

24. The method of claim 23, wherein the ethanol:PG:eucalyptol is present at a ratio of 5:2.5:2.5.

25. The method of claim 22, wherein the MTX is combined with ethanol, PG and santalol.

26. The method of claim 25, wherein the ethanol:PG:santalol is present at a ratio of 5:4:1.

27. The method of claim 22, wherein the MTX is combined with ethanol, PG and ethyl acetate.

28. The method of claim 27, wherein the ethanol:PG:ethyl acetate is present at a ratio of 3:1:1.

29. The method of claim 22, wherein the disorder is selected from the group consisting of scleroderma, rheumatoid arthritis, psoriatic arthritis, lupus, sarcoidosis, Crohn'disease, vasculitis, multiple sclerosis, uterine cancer, lung cancer, head and neck cancer, osteosarcoma, trophoblastic neoplasms, and leukemia, and wherein said administration achieves a plasma concentration of said medicament in said subject that is equivalent to a therapeutic plasma concentration of said medicament delivered via oral or injection route.

30. A composition comprising methotrexate (MTX) in combination with two or more solvents selected from the group consisting of ethanol, transcutol, IPM, migloyl, phosphate buffer (4.0), santalol, eucalyptol, propylene glycol (PG), ethyl acetate, and combinations thereof.

31. The composition of claim 30, wherein the MTX is combined with ethanol, PG and eucalyptol.

32. The composition of claim 31, wherein the ethanol:PG:eucalyptol is present at a ratio of 5:2.5:2.5.

33. The composition of claim 30, wherein the MTX is combined with ethanol PG and santalol.

34. The composition of claim 33, wherein the ethanol:PG:santalol is present at a ratio of 5:4:1.

35. The composition of claim 30, wherein the MTX is combined with ethanol, PG and ethyl acetate.

36. The composition of claim 35, wherein the ethanol:PG:ethyl acetate is present at a ratio of 3:1:1.
